(19) United States
(12) Patent Application Publication
(10) Pub. No.: US 2002/0197630 A1
(43) Pub. Date: Dec. 26, 2002

(54) Systems and methods for high throughput genetic analysis

(76) Inventors: Michael R. Knapp, Redwood City, CA (US); Anne R. Kopf-Sill, Portola Valley, CA (US); Anton Simeonov, Mountain View, CA (US); Christine Peponnet, Sunnyvale, CA (US)

Correspondence Address: CALIPER TECHNOLOGIES CORP, 605 FAIRCHILD DRIVE, MOUNTAIN VIEW, CA, 94043, US

(21) Appl. No.: 10/123,100

(22) Filed: Apr. 11, 2002

Related U.S. Application Data
(60) Provisional application No. 60/283,527, filed on Apr. 12, 2001.

(51) Int. Cl. C12Q 1/68

(52) U.S. Cl. 435/6

(57) ABSTRACT

Methods, devices, libraries, kits and systems for detecting nucleic acids are provided. Universal libraries of standard and modified nucleic acids are provided for nucleic acid detection, as are related methods.

### CROSS REFERENCE TO RELATED APPLICATIONS

This application claims priority to and benefit of U.S. Provisional Patent Application No. 60/283,527, filed on Apr. 12, 2001. The full disclosure of this prior application is incorporated by reference herein.

### STATEMENT REGARDING FEDERALLY SPONSORED RESEARCH OR DEVELOPMENT

The present invention was made with government funding from the United States National Institute of Standards and Technology (NIST), through the Advanced Technology Program (ATP) under Grant No. 70NANB8H4000, and the United States government has certain rights in the invention.

### BACKGROUND OF THE INVENTION

Detection of single nucleotide polymorphisms (SNPs) and other genetic phenomena is an increasingly important technique in molecular biology and medicine. For example, in medical contexts, polymorphism detection is useful for diagnosing inherited diseases and susceptibility to diseases. The detection of SNPs and other polymorphisms can also serve as a basis for tailoring or targeting treatment, i.e., where certain allelic forms of a polymorphism are associated with a response to a particular treatment. In molecular biology, polymorphism detection is fundamental in a variety of contexts, including molecular marker assisted breeding (e.g., of important crop varieties such as Zea and other Graminea, soybeans, etc.), the study of gene diversity, gene regulation and other genetic, epigenetic or para-genetic phenomena.

For example, analysis of polymorphisms in patient genomes has been identified as a potential treasure chest of information about that patient, including that patient's susceptibility to disease, sensitivity to treatments, and the like. Patterns of genetic markers within patient populations are highly instructive in general. Because of this fact, considerable resources have been dedicated to identifying polymorphic markers, as well as to screening patient populations for the presence of previously identified markers.

In general, identification of polymorphic markers, e.g., single nucleotide polymorphisms (SNPs), short tandem repeats (STRs), single base deletions and/or additions, etc. has relied on conventional nucleic acid analysis methods, such as DNA sequencing, denaturing gradient gel electrophoresis, and single strand conformational polymorphism analysis to identify differences in previously known sequences.

The need to screen patient populations for known polymorphic markers, on the other hand, has spawned a number of different technologies that have attempted to increase the throughput, accuracy and robustness of the screening process over traditional, more cumbersome methods such as allele specific oligonucleotide hybridization. Examples of such polymorphism screening techniques include, for example, genetic bit analysis, which involves extension of primer sequences adjacent to a marker sequence. The marker is identified by virtue of the particular base that is incorporated at the marker site, as determined by differential labeling of the primer with dNTPs, e.g., dNTPs having differentially detectable fluorescent labels. Primer extension has also been used to generate extended products that can be analyzed by mass spectrometry. Other techniques include the use of amplification techniques that use primers that overlay the sequence portion that includes the marker. Where the probe hybridizes with the sequence, the sequence is amplified and identified. Absence of the sequence, e.g., resulting from a different base at a marker, results in a failure of hybridization and amplification. Detection can simply involve conventional detection, e.g., electrophoretic separation and detection, or may involve direct reporter systems. Direct amplification and allele scoring has also been accomplished using a version of the fluorogenic Taqman™ probes available from, e.g., Applied Biosystems, Inc., or molecular beacons. Third Wave Technologies, Inc., have proposed a method based upon a similar enzymatic phenomenon, which detects genetic variants by the degradation of an “invader” probe. However, these methods suffer from the same limitation of many of the current technologies for the analysis of genetic variation, namely their high cost. In particular, locus-specific reagents must be prepared for each site of genetic variation. Such fluorescently labeled oligonucleotides can be very costly.

Other methods involve screening a sample material for a variety of different polymorphic markers using large arrays of nucleic acid probes. In particular, a patient sample is washed over large arrays of positionally distinct nucleic acid probes. Identification of the presence or absence of different markers is then accomplished by determining the probes to which the patient sample binds. Nucleic acid arrays are generally commercially available, e.g., GeneChip™ arrays available from, e.g., Affymetrix, Inc. (Santa Clara, Calif.).

While a variety of different methods have been developed for detecting and screening polymorphic markers, these methods are typically useful for screening either a single patient for a large number of markers, also termed loci, e.g., as is the case for arrays, or for screening a large number of patients for a single locus, e.g., as is the case for primer extension or amplification based methods. Accordingly, there still exists a need for screening methods that permit the analysis of relatively large numbers of different patients for relatively large numbers of different polymorphic loci or markers. The present invention meets these and variety of other needs.

### SUMMARY OF THE INVENTION

The present invention relates to methods, devices, probe libraries and systems for performing high-throughput genetic analysis, and in preferred aspects, is directed to such methods, devices and systems for use in genotype analysis of genetic material. For example, the methods, libraries, systems and devices are used to characterize genetic variations that exist in patient populations, e.g., polymorphic genetic markers, e.g., single nucleotide polymorphisms. The identification of such polymorphisms in patients provides for diagnosis, prognosis and treatment options for patients. In the context of non-patient analysis, e.g., plants, non-human animals, fungi, bacteria, cells and the like, the identification of particular polymorphisms provides the basis for selection, e.g., for selection for desirable traits, or against undesirable traits (e.g., in the process of marker-assisted selection).

Accordingly, in a first aspect, the invention provides methods of detecting a target nucleic acid in a sample. The methods include providing at least a first and second group of nucleic acid probes, the first group of probes comprising at least 10% of all possible nucleic acid probes having x number of nucleotides and the second group of probes including at least 10% of all possible nucleic acid sequences having at least y number of nucleotides. At least a first probe from the first group and at least a second probe from the second group is hybridized to target nucleic acid. Hybridization is detected by a non-Sanger detection step (a detection method other than standard Sanger-style sequencing, e.g., other than sequencing using standard incorporation of dideoxynucleic acids), thereby detecting the target nucleic acid. Typically, the first and second probes are substantially proximal when hybridized to the target nucleic acid. This is because proximity of the first probe to the second probe stabilizes binding of the first and second probes to the target nucleic acid.

The size of the probes can be small, which provides ease of synthesis of the nucleic acids. Further, by hybridizing probes in close proximity on a target nucleic acid, small probes can be substantially stabilized in their hybridization. Thus, x or y can be, e.g., an integer between about 5 and about 18, inclusive, e.g., between about 7 and about 12, inclusive, e.g., about 5 to about 10, inclusive, e.g., an integer between about 6 to about 9, inclusive. For example, x or y can be 5, 6, 7, 8, 9, 10, or the like. Optionally, x=y, but x and y can, alternately, be different.

The first and second groups can be components of a single physical group (e.g., the groups can comprise selected members of an array of components), or the first and second groups can be components of a plurality of physical groups (e.g., different arrays of components).

The probes can be natural nucleic acids (e.g., comprising A, G, T, C, or U monomers) or can comprise artificial nucleic acids (LNAs, PNAs, etc.). In one embodiment, the first or second probes comprise at least one promiscuous base. The use of promiscuous bases, particularly in regions of the probe that are not complementary to a target sequence of interest, can reduce the number of probes needed to detect sequences generally, as multiple sequences can hybridize to a single probe comprising one or more promiscuous bases. Example of promiscuous bases include, e.g., inosine, and azidothimidine. Examples of analogs that are optionally components of the groups include nucleobase analogs, sugar analogs, internucleotide analogs and the like. For example, the internucleotide analogs can comprise a phosphate ester analog (e.g., conformationally restricted nucleotides, alkyl phosphonates, phosphoroamidates, alkylphosphotriesters, phosphorothioates, phosphorodithioates, or the like) or a non-phosphate oligonucleotide analog such as a PNA.

In one embodiment, the method comprises hybridizing at least a third group of nucleic acid probes to the target nucleic acid, substantially proximal to at least one of the first and second probes. The third group of nucleic acids comprises at least 10% of all nucleic acid probe sequences having z nucleotides. In general, the first, second or third groups can comprise more than 10% of all possible nucleic acid sequences, e.g., at least 30%, 40%, 50%, 60%, 70%, 80%, 90%, 95% or more of all possible nucleic acid probe sequences having x or y nucleotides. In addition, the first or second groups optionally include probes of length w, where w is not equal to x or y. For example, the length of probes for one or more groups can be selected to normalize Tm of the probes of the group(s) when bound to a target nucleic acid, e.g., the Tₘ of the probes of the first or second group can be selected to be approximately equal. Hybridization can also be regulated by using non standard nucleic acids such as covalently bound minor groove binders or intercalators that enhance hybridization avidity or specificity of the first or second probes to the target nucleic acid, or by using non-standard monomers to construct a probe of interest, e.g., a PNA, LNA or the like. The first, second and third probes can be ligated together, e.g., using a ligase. Because the ligation reaction preferentially occurs between adjacently bound probes, detection of the ligation (e.g., via a nucleic acid size detection method) provides an additional detection mechanism useful in the present invention. A ligated probe can also be used in subsequent hybridization reactions.

In several embodiments, the probes from the first, second and/or third groups are labeled with a label. In one class of embodiments, the label of the probes from the second group is different from the label of the probes from the third group, providing for detection of multiple labels simultaneously, e.g., via label interaction effects (e.g., colorimetric or fluorescent interactions). For example, in one embodiment, the first and second probes comprise a FRET pair and detecting hybridization of the first or second probes comprises fluorescence resonance energy transfer (FRET) detection. For example, the first probe is optionally labeled with a fluorescent reporter moiety, and the second probe is optionally labeled with a quencher moiety, such that upon hybridization of the probes with the target nucleic acid, fluorescence of the reporter moiety is quenched, thereby reducing fluorescence of the reporter moiety. Alternately, in one embodiment, the first probe is labeled with a fluorescent reporter moiety, e.g., at one of its termini, and the second probe is labeled with a quencher moiety, e.g., at one of its termini, such that hybridization of the first and second probes with the target nucleic acid causes an increase in fluorescence emission. In one other example, the first or second probe is labeled with a fluorescent reporter dye at one of its termini, and the third probe is labeled with a quencher molecule at one of its termini, such that upon hybridization of the probes from the first, second and third groups with the sample, the fluorescence of the reporter dye is quenched so as to cause a reduction in fluorescence emission of the reporter dye. Examples of useful fluorescent reporter moieties include Xanthene dyes, Cyanine dyes, and metal-ligand complexes. A variety of FRET pairs are noted herein and can be used in the invention, including, e.g.,: terbium chelate and TRITC (tetrarhodamine isothiocyanate), europium cryptate and Allophycocyanin, DABCYL and EDANS, Fluorescein and Tetramethylrhodamine, IAEDANS and Fluorescein, Fluorescein and Fluorescein, BODIPY FL and BODIPY FL, and Fluorescein and QSY 7 dye. The detecting step optionally comprises observing the fluorescence of the hybridized probes while varying temperature over a range of temperatures.

In one class of embodiments, the range of temperatures during which fluorescence or other label detection events are observed is conducted in a range of from about 0° C. to about 60° C. The temperature can be modified (e.g., swept) between different temperatures during detection. In one class of embodiments, the detecting step comprises measuring a signal intensity resulting from hybridization of the hybridizing probes and the target nucleic acid (e.g., optionally by monitoring the signal intensity during a temperature sweep). This detection can be performed, e.g., in a microfluidic device, e.g., by providing an analysis channel in a microfluidic device that comprises one or more detection regions and one or more temperature control regions.

In one significant class of embodiments, the target nucleic acid comprises a polymorphic variant sequence. In one typical detection format, the first probe is fully complementary to the polymorphic variant sequence and the second probe hybridizes substantially adjacent to the probe hybridized to the polymorphic variant sequence. Thus, detecting the target nucleic acid optionally comprises detecting the polymorphic variant sequence.

The target nucleic acid can be any relevant nucleic acid, e.g., a biological nucleic acid, e.g., derived from a patient, an animal (a human or non-human animal), a plant, a bacteria, a fungi, an archae, a cell, a tissue, an organism, etc. For example, where the target nucleic acid is derived from a bacteria, archae, plant, non-human animal, cell, fungi, or non-human organism, the method optionally further comprises selecting the bacteria, archae, plant, non-human animal, cell, fungi, or non-human organism based upon detection of the target nucleic acid. In one embodiment, the target nucleic acid is derived from a patient, e.g., a human patient. In this embodiment, the method optionally further includes selecting a treatment, diagnosing a disease, or diagnosing a genetic predisposition to a disease, based upon detection of the target nucleic acid.

In one class of typical embodiments, the first and second probes are hybridized to the target nucleic acid in a mixture comprising a buffer. Components of the buffer, such as salt can be used to control hybridization parameters (increasing salt concentration is one way of decreasing hybridization stringency). For example, the buffer can comprise salt in a concentration from a range of about 0.2M to about 2M, e.g., in the range of about 0.5M to about 1.5M, e.g., in a range of about 0.8M to about 1.2 M, e.g., about 1M.

In a related embodiment, methods for detecting a polymorphic variant in a polymorphic nucleic acid sequence are provided. In the methods, a mixture comprising a polymorphic nucleic acid sequence, at least two probes and a buffer is flowed into an analysis channel, one of the at least two probes being complementary to a portion of the polymorphic nucleic acid sequence comprising the polymorphic variant, and the other probe being complementary to a substantially adjacent portion of the polymorphic nucleic acid sequence. Hybridization of at least one of the at least two probes is detected to determine the identity of the polymorphic variant in the polymorphic nucleic acid sequence, by varying temperature within a detection region located at a position along a length of the analysis channel.

In an additional related embodiment, the invention provides methods of detecting a target nucleic acid. In the methods, a mixture comprising the target nucleic acid is flowed into an analysis channel. At least a first probe and a second probe are also flowed into the analysis channel. The first probe is hybridized to the target nucleic acid. The second probe is also hybridized to the target nucleic acid. Hybridization of the second probe to the target nucleic acid substantially adjacent to the first probe stabilizes hybridization of the first probe. Hybridization is detected via a non-Sanger detection step (using detection other than standard dideoxy sequencing).

For both of these related embodiments, all of the above description of probe lengths, presence of percentages of total possible nucleic acids of a given sequence in source probe groups, use of multiple probe groups (e.g., 1, 2, or 3 or more probe groups) selection of probe groups, buffer conditions, use of the method on particular nucleic acids, detection of different labels, FRET and other signal detection, determination of signal intensities, use in microfluidic systems, use of nucleic acids and analogs, selection of probes with approximately equal or different Tₘs, temperature sweeps, buffer concentrations, and in every other applicable aspect, apply equally to these related embodiments.

The present invention also provides related sets of nucleic acid probes for the detection of a target nucleic acid sequence in a sample. The set includes at least two groups of nucleic acid probes, a first of the at least two groups comprising at least 10% of all possible nucleic acid probe sequences having x nucleotides, and a second of the at least two groups comprising at least 10% of all possible nucleic acids having y nucleotides. Typically, a plurality of members of each of the first and second groups are labeled.

Here again, the issues noted above for various methods of the invention are applicable to the compositions of the invention. For example, labels of the members of the first group can interact with labels of the second group, e.g., when the labels are in proximity to one another (e.g., via FRET, colorimetric labeling, or the like). For example, the probes of the first group optionally comprise a first label and the probes of the second group comprise a second label, where the first label comprises an acceptor FRET moiety and the second label comprises a donor FRET moiety. For example, the acceptor moiety can incldue a quencher moiety such as a fluorophore, Dabsyl, Black-hole™, QSY™, an Eclipse Dark Quencher, or the like. The donor can be, e.g., a Xanthene dye, a Cyanine dye, a Metal-Ligand Complex, a Coumarin dye, a BODIPY dye, a Pyrene dye, or the like. A variety of FRET pairs are noted herein, including: terbium chelate and TRITC (tetrarhodamine isothiocyanate), europium cryptate and Allophycocyanin, DABCYL and EDANS, Fluorescein and Tetramethylrhodamine, IAEDANS and Fluorescein, Fluorescein and Fluorescein, BODIPY FL and BODIPY FL, and Fluorescein and QSY 7 dye.

In preferred embodiments, hybridization of a member of the first group to the target nucleic acid stabilizes hybridization of a member of the second group. The two groups can include at least 20%, 30%, 40%, 50%, 60%, 70%, 80%, 90% 95% or more of all possible nucleic acid probe sequences for nucleic acid probes of length x or y. In one class of embodiments, the first or second groups comprise probes of a length other than x or y. For example, at least one of the at least two groups optionally comprise a subset of probes having w number of nucleotides, wherein when present in the first set, w is not equal to x and when present in the second set w is not equal to y. This is useful in embodiments where probe sets are selected to have approximately similar Tₘs, e.g., to account for AT or GC content of the various probes. The probe set can also include a third group of nucleic acids, e.g., including at least 10% of all nucleic acid probe sequences having z nucleotides. The first second and third groups are optionally components of a single physical group, but can also be components of different physical groups.

As described above in the context of the various methods, x, y, z and w can all represent lengths of e.g., between about 1 and about 18, inclusive. For example, in one class of embodiments, w is an integer between about 1 and about 10, inclusive, or is an integer between about 1 and about 8, inclusive. Similarly, in one embodiment, z is an integer between about 5 and about 10, inclusive. In one embodiment, x is an integer between about 5 and about 10, inclusive, or e.g., an integer between about 6 and about 9, inclusive, e.g., 7. Similarly, y can be an integer between about 5 and about 18, inclusive, or e.g., an integer between about 7 and about 12, inclusive, e.g., 10. Optionally, x=y=z, but x, y and z can all be different as well.

In a related aspect, the invention provides a library of nucleic acids. The library includes at least about 10% of all possible nucleic acids for a monomer length x, e.g., where x is greater than or equal to 5. In one aspect, the nucleic acids comprise non-natural nucleic acid monomers (e.g., PNAs, LNAs, or the like).

The library can include any of the artificial monomers noted herein or which are generally available, e.g., monomers that make up a PNA, an LNA, a base-modified nucleic acid, a nucleobase analog, a sugar analog, an internucleotide analog, or the like. Typically, at least 90% of the 10% of all possible nucleotides comprise of one or more artificial monomer such as a PNA monomer, an LNA monomer, a base-modified nucleic acid monomer or the like.

The library can include at least about 10% of all possible nucleic acids for a monomer length y, wherein y is greater than or equal to 5 and does not equal x, and wherein the nucleic acids comprise non-natural nucleic acid monomers. Similarly, the library can include at least about 10% of all possible nucleic acids for a monomer length z, wherein z is greater than or equal to 5 and does not equal x or y, and wherein the nucleic acids comprise non-natural nucleic acid monomers. The issues noted above for inclusion of probes of different lengths in any applicable probe set (e.g., probes of length w) apply here as well.

Where the library comprises non-natural nucleic acids, the nucleic acids of the library optionally display greater avidity or specificity for a target nucleic acid than a corresponding natural nucleic acid (a nucleic acid that includes only the standard bases typically found in naturally occurring biological nucleic acids, e.g., A, T, U, C, G and minor variants thereof).

Typically, the nucleic acids of the library comprise one or more labels, e.g., one or more fluorescent, luminescent, or colorimetric labels. Indeed, any of the labeling schemes noted herein can be applied to this embodiment of the invention, e.g., where the labels of the library comprise one or more FRET pairs, or the like.

The format of the library can vary. In one embodiment, members of the library are arranged in substantially separate pools (e.g., less than about 10 probes per pool, typically less than 5 probes per pool, e.g., approximately one probe per pool). In another, the members of the library are arranged in substantially overlapping pools (e.g., more than about 10 probes per pool). Substantially separate pools provides the advantage of simplified data deconvolution, as the contents of any hybridization reaction can be known in advance. The use of overlapping pools provides for higher density reagent storage and access, but typically involves a data deconvolution operation.

The physical format of the pools can also vary. For example, in one embodiment, the members of the library are arranged dried on a solid surface in a re-hydrateable form. In another, the members of the library are arranged in liquid storage elements such as microtiter wells, microfluidic chambers, channels, or the like. The members of the library can be arranged in standard laboratory system formats, or can be arranged in a microfluidic system, or in a format accessible by a microfluidic system.

In another aspect, the invention includes a genetic analysis system. The system includes a vessel comprising a mixture that includes a target nucleic acid. The system also typically includes a plurality of sources of nucleic acid probes, the plurality of sources each including probes of at least 10% of all possible nucleic acid probe sequences of length x or y. The genetic analysis system also includes a subsystem that selectively delivers different probes from the plurality of sources of probes to the vessel (e.g., a microfluidic device). This subsystem includes, e.g., system instructions which identify and select probes to be delivered to the vessel, and a sampling system for sampling and delivering probes from the plurality of sources of probes to the vessel. Typically, the vessel is a microfluidic device, and the system instructions select probes that are complementary to a region of interest on the target nucleic acid. All of the above noted optional arrangements of probes, libraries, storage systems and every other applicable feature here as well.

In one preferred embodiment, the sampling system comprises a pipettor affixed to the microfluidic device. The operation of sampling and delivering probes can incldue delivering at least one, two, three or more nucleic acid probes from the plurality of sources of probes to the vessel. The nucleic acid probes typically comprise hybridizing probes and flanking sequence probes, where the hybridizing probes comprising at least one interrogation base (a base that hybridizes, or does not hybridize to a target at a sequence of interest).

The probes can include any of the artificial monomer types noted herein, e.g., nucleobase analogs, sugar analogs, internucleotide analogs, etc. in the sequence of the probes. As above, the nucleobase analogs optionally include covalently bound minor groove binders, intercalators or other modifications for enhancing hybridization avidity or specificity of the nucleic acid probes. For example, nucleobase analogs can include non-covalently bound minor groove binders such as DAPI, or Hoeschst 33258. The internucelotide analogs optionally comprise a phosphate ester analog (e.g., alkyl phosphonates, phosphoroamidates, alkylphosphotriesters, phosphorothioates, phosphorodithioates, etc.), or a non-phosphate oligonucleotide analog.

As with any of the other embodiments herein, the probe sources can include any percentage of total possible sequences for x or y that are noted herein (e.g., anything from 5% to 100% of possible sequences). For example, the plurality of sources of nucleic acid probes optionally comprise sources of at least 75%, 85%, or 95% or more of all possible nucleic acid probe sequences of length x or y (or z or w, when such additional probe sets are present in the system). Any of the size ranges noted above for the probes apply to the system as well, as do any of the buffer conditions for the mixture.

The vessel is optionally in contact with a thermal element (an element that regulates temperature), whereby at least a region of the vessel is subjected to an increase or decrease in temperature. The system optionally further includes a signal detector, such as a fluorescent emission detector.

The microfluidic device optionally includes at least two intersecting microscale channels, e.g., where at least one of the at least two intersecting channels is an analysis channel that can be subjected to an increase or decrease in temperature.

### BRIEF DESCRIPTION OF THE FIGURES

FIG. 1 schematically illustrates hybridization-based detection of single nucleotide polymorphisms.

FIG. 2 schematically illustrates a hybridization based discrimination method that utilizes stacked probes to discriminate the particular polymorphic marker at a given locus.

FIG. 3 schematically illustrates a temperature profile of hybridization for a molecule that is either perfectly matched with the target sequence or which includes a single base mismatch.

FIG. 4 schematically illustrates different thermal sweeps for perfectly matched and single base mismatched hybridizations of AT rich sequences (FIG. 4A) and GC rich sequences (FIG. 4B).

FIG. 5 illustrates use of anchor probe systems for hybridization based detection of matched and mismatched hybridizations.

FIG. 6 schematically illustrates an example of software/system instruction steps for the selection of probes for use in the methods of the present invention.

FIG. 7 schematically illustrates an overall system for carrying out the present invention.

FIG. 8 schematically illustrates an example of a channel network in a microfluidic device, for carrying out a high throughput embodiment of the invention.

FIG. 9 panels A and B illustrate detection by fluorescence polarization of a perfect match between a LNA probe and target sequence. FIG. 9A shows Locus 213 PCR products genotyped with LNA 601 and FIG. 9B shows Locus 213 PCR products genotyped with LNA 602.

### DEFINITIONS

A “target nucleic acid” is a nucleic acid to be detected. The target nucleic acid can be isolated from a natural source or can be an amplified (e.g., PCR amplified) sequence.

“Oligonucleotide” and “polynucleotide” are used interchangeably and mean single stranded and/or double-stranded polymers of nucleotide monomers, e.g., monomers linked by internucleotide phosphodiester bond linkages or inter nucleotide analog linkages.

A “nucleotide” refers to a phosphate ester of a nucleoside.

A “nucleoside” refers to a compound consisting of a nucleobase linked to the C-1 carbon of a ribose sugar.

As used herein, the term “adjacent” is used when two or more probes are next to each other, when there is no intervening nucleotides between the two, e.g., when hybridized to a target nucleic acid.

In a related context, the term, “substantially adjacent” refers to positioning of two probes with a gap of less than six, generally less than 4, often less than 3 or less than 2 intervening nucleotides between them, e.g., when hybridized to a target sequence.

The term “coaxial stacking” generally refers to hybridization of two or more probes to immediately adjacent stretches of target sequence.

A “nucleic acid probe” comprises an oligonucleotide or analog thereof (e.g., an oligonucleotide of standard nucleotides, or PNA, or the like) that is capable of forming a duplex structure by complementary pairing with a sequence of a target nucleic acid and which comprises a detectable signal.

An “interrogation base” is a base location within an “interrogating probe” that is complementary to a position comprising the polymorphic variant on a target nucleic acid sequence.

An “anchor” as used herein comprises a complementary nucleic acid probe or a flanking sequence that, when hybridized to a target nucleic acid sequence, is complementary to a region adjacent to the region complementary to an interrogating probe.

A “microfluidic device” is an apparatus or component of an apparatus having microfluidic reaction channels and/or chambers. Typically, at least one reaction channel or chamber will have at least one cross-sectional dimension between about 0.1 μm and about 500 μm.

A “reaction channel” is a channel (in any form, including a closed channel, a capillary, groove, a receptacle or the like) on or in a microfluidic substrate.

A “reagent channel” is a channel (in any form, including an enclosed channel, a capillary, a groove, a receptacle or the like) on or in a microfluidic substrate, through which components are transported.

As used herein a “temperature sweep” refers to varying temperature of a region between a range of temperatures. e.g., to perform a reaction step or to detect reaction products within the region.

A “non-Sanger” detection step is a detection step that operates by a mechanism other than standard Sanger dideoxy nucleic acid sequencing. Examples include direct detection of hybridization (e.g., FRET based detection, FP, molecular beacon detection, TaqMan™ detection, mobility shift assays, etc.) and sequencing methods other than standard dideoxy sequencing (e.g., sequencing by incorporation, Maxam-Gilbert sequencing, etc.). In certain embodiments, the invention includes the use of “non-enzyme mediated” detection methods, e.g., methods that do not require ligase or polymerase to operate.

The phrase “all possible nucleic acids” in the context of a percentage of possible nucleic acid sequences in a set (e.g., 10% of all possible sequences) is meant with reference to a standard base code (A, T or U, G and C), regardless of the actual nucleic acid monomer type. Thus, one or more of the probe monomers can include a non-natural monomer, e.g., a PNA or LNA monomer, but sequences that are complementary for hybridization purposes are considered as contributing a single sequence to the relevant percentage of possible sequences. Thus, if a probe set includes both a PNA sequence that is complementary to the sequence ATGCAC in a target nucleic acid and a standard nucleic acid having the sequence TACGTG, the two probes which differ by type (PNA vs. standard nucleic acid) are not considered to differ by sequence for purposes of calculating the percentage of possible nucleic acid sequences in the probe set. That is, the probes together count as a single sequence entry, unless inappropriate to the context at issue. Thus, the total number of sequences that make up all possible 6 mers is considered to be 4,096, even though a given set of 6 mers optionally has different types of probes that actually display a greater number of probes than sequences (e.g., a probes set of 6 mers can include, e.g., 4,096 standard nucleic acid probes, plus 4,096 PNA probes, providing 8,192 total probes covering 4,096 sequences). Similarly, bases that hybridize to the same partner (e.g., T and U both hybridize to A) are typically only counted once, unless indicated otherwise in a particular context.

### DETAILED DESCRIPTION OF THE INVENTION

I. General

The present invention generally relates to high-throughput systems and methods for screening relatively large numbers of different sample materials for relatively large numbers of different genetic loci or marker sequences. The present invention combines the high-throughput, automatability, integratability and miniaturization of microfluidic analytical systems with novel biochemistries that are useful, e.g., for genetic marker analysis. Thus, the invention is directed to systems, libraries and process that are used, e.g., in genetic analysis, as well as to novel individual elements of those systems and processes.

In general, the present invention is directed to methods and systems that employ hybridization based detection methods to ascertain the presence of polymorphic genetic markers, for example, SNPs, STRs, deletions, insertions, etc., within one or more target nucleic acid sequences. The hybridization methods of the present invention typically employ short probe sequences of nucleotides and/or nucleotide analogs, to identify whether a polymorphic sequence is present in the target sequence. By employing shorter probes, and operating on a readily automatable platform, e.g., microfluidics, one can utilize a probe library (e.g., a complete or substantially complete library) to screen for sequences of interest, in a high-throughput format.

By selecting the nature of the probes to be used, e.g., one or more stacked probes, standard nucleic acid probes, nucleic acid analog probes and/or mixed probes of nucleic acids and nucleic acid analogs, one can improve the discrimination capability of these shorter probes in identifying a particular polymorphic variant.

Further, depending upon the nature of the hybridization operation, one can also select the hybridization conditions, and select from detection methods that substantially improves one's ability not only to detect the presence or absence of a hybridization reaction that might indicate the presence of a polymorphic variant, but that also increase one's confidence in the accuracy of that determination.

Yet another advantage of using a microfluidic platform for performing the hybridization is the feasibility of performing a thermal sweep to detect the strength of the hybridization by measuring signal intensities for a plurality of hybridized probes in a high throughput format.

Accordingly, in a first aspect, the present invention provides methods for detecting a target nucleic acid sequence in a sample by providing at least two groups of nucleic acid probes. The first group of probes including at least 10% of all possible nucleic acid probes having x number of nucleotides and the said second group of probes including at least 10% of all possible nucleic acid sequences having at least y number of nucleotides. The methods involve hybridizing at least a first probe from the first group and at least a second probe from the second group to target nucleic acid and detecting the hybridization of the probe with the target nucleic acid. When hybridized to the target nucleic acid, the first and second probes are substantially proximal, wherein proximity of the first probe to the second probe stabilizes binding of at least one of the first and second probes. In a preferred embodiment, x is an integer within 5 to 10, for example, an integer within 6 to 9, for example x equals 7 nucleotides. Y can be an integer between 5 and 18 nucleotides, inclusive, for example between 7 and 12, inclusive, for example, y can equal 10 nucleotides, for example. In some situations, x can be the same as y or an integer different from y. This is particularly useful where the probes are normalized to approximately the same Tₘ, e.g., to account for differences in A/T or G/C content. The target nucleic acid may be derived from a patient, and the method can comprise selecting a treatment diagnosing a disease or diagnosing a genetic predisposition to a disease based upon detection of the target nucleic acid. The target nucleic acid may also derived from a bacteria, archae, plant or animal, for example, and the method can comprise selecting the bacteria, archae, plant or animal based upon detection of the target nucleic acid.

The first and second groups of probes can be components of a single physical group, or can be components of a plurality of physical groups. The first or second probes can comprise at least one promiscuous base. For example, the at least one promiscuous base is selected from a group consisting of: inosine and azidothimidine. The first or second probes can comprise one or more of: a nucleobase analog, a sugar analog or an internucleotide analog. The nucleobase analogs can include covalently bound minor groove binders or intercalators that enhance hybridization avidity or specificity of the nucleic acid probes to a target. The internucleotide analogs can comprise one or more of: a phosphate ester analog and a non-phosphate oligonucleotide analog, for example. For example, the non-phosphate oligonucleotide analog is a PNA. The phosphate ester analogs can be selected from a group consisting of conformationally restricted nucleotides, alkyl phosphonates, phosphoroamidates, alkylphosphotriesters, phosphorothioates and phosphorodithioates.

In the method of detection of the probes to the target nucleic acid sequence, the at least one probe from the first group can be labeled with a fluorescent reporter moiety at one of its termini, and the at least one probe from the second group can be labeled with a quencher moiety at one of its termini, such that upon hybridization of the probes from the first and second groups with the target nucleic acid sequence, the fluorescence of the reporter moiety is quenched so as to cause a reduction in fluorescence of the reporter moiety. Alternatively, the at least one probe from the first group can be labeled with a fluorescent reporter moiety at one of its termini, and the at least one probe from the second group can be labeled with a quencher moiety at one of its termini, such that hybridization of the probes from the first and second groups with the target nucleic acid sequence causes an increase in fluorescence emission. The step of detecting hybridization of the probes can then comprise detection by fluorescence resonance energy transfer (FRET), wherein the probe from the first group and the probe from the second group comprise a FRET pair. The fluorescent reporter moiety can be selected from a group consisting of: Xanthene dyes, Cyanine dyes, Metal-Ligand Complexes, for example. The detecting step can further comprise observing the fluorescence of the reaction mixture while varying temperature over a range of temperatures, wherein the range of temperatures is from about 0° C. to about 80° C, for example between about 0° C. to about 60° C.,

In one aspect of the invention, the first and second groups of probes each include at least 60% of all possible nucleic acid probe sequences of length x and y respectively (where x can equal y, for example), for example, the first and second groups can each include at least 70% of all possible nucleic acid probe sequences of length x and y respectively, at least 80% of all possible nucleic acid probe sequences of length x and y respectively, for example at least 90% of all possible nucleic acid probe sequences, for example at least 95% of all possible nucleic acid probe sequences of length x and y, respectively. In a presently preferred embodiment, the method can comprise at least a third group of nucleic acid probes including at least 10% of all nucleic acid probe sequences having z nucleotides, wherein the method further comprises hybridizing at least one third probe from the third group to the target nucleic acid sequence substantially proximal to at least one of the first and/or second probes. In such method, the probes the second and third groups may each be labeled with a label, wherein the label of the probes from the second group may be different from the label of the probes from the third group. For example, at least one probe from the second group is labeled with a fluorescent reporter dye at one of its termini, and at least one probe from the third group is labeled with a quencher molecule at one of its termini, such that upon hybridization of the probes from the first, second and third groups with the sample target nucleic acid sequence, the fluorescence of the reporter dye is quenched so as to cause a reduction in fluorescence emission of the reporter dye.

The teachings of the present invention can further be used to determine a polymorphic variant in a polymorphic variant nucleic acid sequence. In such situation, the first probe is fully complementary to the polymorphic variant sequence and the second probe hybridizes substantially adjacent to the polymorphic variant sequence and the step of detecting the target nucleic acid comprises detecting the polymorphic variant sequence.

In a related aspect, the present invention provides a set of nucleic acid probes for detection of a target nucleic acid sequence in a sample, comprising at least two groups of nucleic acid probes, a first of the at least two groups comprising at least 10% of all possible nucleic acid probe sequences having x nucleotides, and a second of the at least two groups comprising at least 10% of all possible nucleic acids having y nucleotides, wherein a plurality of members of each of the first and second groups are labeled. The labels of the members of the first group interact with labels of the second group, when the labels are in proximity to one another, such that hybridization of a member of the first group to the target nucleic acid stabilizes hybridization of a member of the second group. As above, the two groups can each include at least 60% of all possible nucleic acid probe sequences for nucleic acid probes of length x or y, for example at least 70% of all possible nucleic acid probe sequences for nucleic acid probes of length x or y. For example at least 80% of all possible nucleic acid probe sequences for nucleic acid probes of length x or y, for example at least 90% of all possible nucleic acid probe sequences for nucleic acid probes of length x or y, for example at least 95% of all possible nucleic acid probe sequences for nucleic acid probes of length x or y. The probes of the first group can comprise a first label and the probes of the second group comprise a second label, wherein, for example, the first label comprises an acceptor FRET moiety and the second label comprises a donor FRET moiety. The acceptor moiety comprises a quencher moiety, wherein the quencher moiety can be selected from a group consisting of: fluorophores, Dabsyl, Black-hole™, QSY™, Eclipse Dark Quencher, for example. The donor moiety may be selected from a group consisting of: Xanthene dyes, Cyanine dyes, Metal-Ligand Complexes, Coumarin dyes, BODIPY dyes, and Pyrene dyes, and the like.

In another aspect of the present invention, there is provided a method for detection of a polymorphic variant in a polymorphic nucleic acid sequence by hybridization, the method comprising flowing a mixture comprising a polymorphic nucleic acid sequence, at least two probes and a buffer into an analysis channel, one of said at least two probes being complementary to a portion of the polymorphic nucleic acid sequence comprising the polymorphic variant, and the other probe being complementary to a substantially adjacent portion of the polymorphic nucleic acid sequence; and detecting hybridization of at least one of the at least two probes to determine the identity of the polymorphic variant in the polymorphic nucleic acid sequence by varying temperature within a detection region located at a position along a length of the analysis channel. The mixture buffer preferably comprises Salt in a concentration from a range of about 0.2M to 2M, for example in the range of about 0.5M to 1.5M, for example in the range of about 0.8M to 1.2 M, for example the Salt concentration is about 1M. The detecting step further comprises measuring a signal intensity from hybridization of the hybridizing probes and the polymorphic nucleic acid sequence. The analysis channel is optionally provided in a microfluidic device, wherein the analysis channel comprises one or more detection regions and one or more temperature control regions.

In a further aspect of the present invention, a genetic analysis system is provided which comprises a vessel having disposed therein a mixture, said mixture comprising a target nucleic acid; a plurality of sources of nucleic acid probes, the plurality of sources each including probes of at least 10% of all possible nucleic acid probe sequences of length x or y (wherein x and y is an integer between 5 to 10, inclusive, for example between 6 and 9, for example 6 or 7 nucleotides); selectively delivering different probes from the plurality of sources of probes to the vessel, comprising: a computer program for identifying and selecting probes to be delivered to the vessel; and a sampling system for sampling and delivering probes from the plurality of sources of probes to the vessel, wherein the vessel is a microfluidic device, and the computer program selects probes that are complementary to region of interest on the target nucleic acid. The sampling system can comprises a pipettor affixed to the microfluidic device, for example. The nucleic acid probes can comprise hybridizing probes and flanking sequences, the hybridizing probes comprising at least one interrogation base, for example. The plurality of sources of nucleic acid probes can comprise sources of at least 30% of all possible nucleic acid probe sequences of length x or y, for example at least 75% to 85% of all possible nucleic acid probe sequences of length x or y, for example at least 95% of all possible nucleic acid probe sequences of length x or y. The reaction vessel preferably is in contact with a thermal element whereby at least a region of the reaction vessel is subjected to an increase or decrease in temperature. microfluidic device comprises at least two intersecting microscale channels wherein at least one of said at least two intersecting channels is a reaction channel.

In another aspect of the present invention, a method of detecting a target nucleic acid is disclosed which comprises flowing a mixture comprising the target nucleic acid in an analysis channel; flowing at least a first probe and a second probe into said analysis channel hybridizing a first probe to the target nucleic acid; hybridizing a second probe to the target nucleic acid, wherein the second probe hybridizes to the target nucleic acid adjacent to the first probe, and wherein hybridization of the second probe stabilizes hybridization of the first probe; and detecting hybridization of the first probe by a non-Sanger detection step. The target nucleic acid may be derived from a patient, an animal, a plant, a bacteria, a fungi, an archae, a cell, a tissue, or an organism, for example. The target nucleic acid may comprise a polymorphic sequence. The first and/or second probe cam comprise a fluorescent label, for example, the first probe comprises a first label and the second probe comprises a second label, wherein the first label is quenched by proximity to the second label such that the first probe and the second probe collectively comprise a FRET label pair. Detection of hybridization of the first probe then comprises detecting FRET between the first label on the first probe and the second label on the second probe. The first and/or second probe may be provided from at least one probe set comprising at least 10% of all possible nucleic acids of a selected type for a selected length, wherein the selected length is at least 5 probe monomers which comprise, for example, one or more of: a nucleotide and a PNA monomer, for example, the first probe is provided from at least one probe set comprising at least 30% of all possible nucleic acids of a selected type for a selected length, wherein the selected length is at least 5 probe monomers and the second probe is provided from at least a second probe set comprising at least 30% of all possible nucleic acids of a selected type for a selected length, wherein the selected length is at least 5 probe monomers. The first and second probes may be components of a single physical group or may be components of multiple physical groups.

Additional details on the methods, systems, libraries, and the like are found below.

II. Methods

As noted previously, the methods, devices and systems of the invention benefit from advances in integration, automation and miniaturization brought on by advances in microfluidic technology. In preferred aspects, the entire screening method or a substantial subset of the entire operation, from sample preparation to discrimination and detection, is carried out in a single integrated microfluidic channel network.

A. Sample Preparation

As with most genetic analyses, a number of operations precede the actual analytical or detection step. These operations are generally referred to as sample preparation operations and typically include harvesting a target nucleic acid from its origin such as the cells of a patient, a plant, an animal, a culture, or the like, which can involve, e.g., cell lysis, separation of cellular debris from the soluble fraction, and purification or partial purification of the nucleic acids. Typically, genetic analyses also employ an amplification step where, because of the relatively low concentrations of a given nucleic acid sequence in a cell, the target nucleic acid is selectively replicated or amplified to levels that facilitate its detection. It will be appreciated that purification of nucleic acids can often be dispensed with, in that thermal amplification protocols exist which directly amplify nucleic acids from source materials with a minimum of pre-amplification preparative steps. A number of different methods have been described for amplifying nucleic acids including the polymerase chain reaction (PCR), and the ligase chain reaction (LCR).

General texts which describe isolation, synthesis, cloning and amplification of nucleic acids from biological sources, including the use of vectors, promoters and many other relevant topics, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2000) (“Ausubel”)). Examples of techniques sufficient to direct persons of skill through general in vitro amplification methods, including the polymerase chain reaction (PCR) the ligase chain reaction (LCR), Qβ-replicase amplification and other RNA polymerase mediated techniques (e.g., NASBA), e.g., for the production or isolation of the nucleic acids of the invention (whether targets or probes) are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369: 684-685 and the references therein, in which PCR amplicons of up to 40 kb are generated.

Other useful references, e.g. for cell isolation and culture (e.g., for subsequent nucleic acid isolation) include Freshney (1994) Culture of Animal Cells a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla.

The polynucleotides of the invention (particularly probes) can also be prepared by chemical synthesis using, e.g., the classical phosphoramidite method described by Beaucage et al., (1981) Tetrahedron Letters 22:1859-69, or the method described by Matthes et al., (1984) EMBO J. 3: 801-05, e.g., as is typically practiced in automated synthetic methods. According to the phosphoramidite method, oligonucleotides are synthesized, e.g., in an automatic DNA synthesizer, and, optionally purified, annealed, ligated, cloned amplified or otherwise manipulated by standard methods to produce additional nucleic acids.

The modifications to such protocols to accommodate non-natural monomers such as PNAs or LNAs are also well known. For LNAs, see also, proligo.com; Koshkin et al. (1998) Tetrahedron 54:3607-3630; Koshkin et al. (1998) J. Am. Chem. Soc. 120:13252-13253; Wahlestedt et al. (2000) PNAS. 97:5633-5638. For PNAs see also, bostonprobes.com; and Buchardt et al. (1993) “Peptide nucleic acids and their potential applications in biotechnology” TIBTECH. 11:384-386; Corey (1997) “Peptide nucleic acids: expanding the scope of nucleic acid recognition” TIBTECH 15:224-229; Dueholmand and Nielsen (1997) “Chemistry, properties and applications of PNA” New J. Chem. 21:19-31; Hyrup and Nielsen “Peptide Nucleic Acids (PNA): Synthesis, Properties and Potential Applications” Bioorg. Med. Chem. 4:5-23; Nielsen et al. (1994) “Peptide Nucleic Acid (PNA). A DNA mimic with a peptide backbone” Bioconjugate Chemistry 5:3-7; Nielsen (1995) “DNA analogues with nonphosphodiester backbones” Annu. Rev. Biophys. Biomol. Struct. 24:167-183; Nielsen et al. (1993) “Peptide nucleic acids (PNA): oligonucleotide analogs with a polyamide backbone” Antisense Research and Applications (eds Crooke and Lebleu) 364-373 CRC Press; Nielsen (1999) “Peptide nucleic acid. A molecular with two identities” Acc. Chem. Res. 32: 624-630; Ørum et al. (1997) “Peptide Nucleic Acid” Laboratory Methods for the Detection of Mutations and Polymorphisms in DNA Chapter 11 (ed. Taylor, G. R.) 123-133 (1997); and Ørum et al. (1997) “Peptide Nucleic Acid” Nucleic Acid Amplification Technologies: Applications to Disease Diagnostics. (ed. Lee et al.) pp. 29-48.

In addition, essentially any nucleic acid (and virtually any labeled nucleic acid, whether standard or non-standard) can be custom or standard ordered from any of a variety of commercial sources, such as The Midland Certified Reagent Company (mcrc@oligos.com), The Great American Gene Company (http://www.genco.com), ExpressGen Inc. (www.expressgen.com), Operon Technologies Inc. (Alameda, Calif.) and many others. PNAs are generally commercially available, e.g., from the Applied Biosystems Division of the Perkin-Elmer Corporation (Foster City, Calif.). PNAs are also available, e.g., from Boston Probes Inc. (Bedford, Mass.). LNAs are available, e.g., from Proligo LLC (Boulder, Colo.).

As noted, essentially any nucleic acid or nucleic acid analogue can be used in the context of the present invention, including DNAs, LNAs, RNAs, PNAs and analogues thereof. One of skill will be fully aware of many different analogues and methods for making such analogues. Additional details on certain analogues, including certain nuclease resistant analogues, are found in e.g., Egholm, M. et al., (1993) Nature 365:566-568; Perry-O'Keefe, H. et al., (1996) Proc. Natl. Acad. USA 93:14670-14675; Miller, et al., “Nonionic nucleic acid analogues. Synthesis and characterization of dideoxyribonucleoside methylphosphonates”, Biochemistry 1979, 18, 5134-5143. Divakar, et al., “Approaches to the Synthesis of 2′-Thio Analogues of Pyrimidine Ribosides”, J. Chem. Soc., Perkins Trans., I, 1990, 969-974; U.S. Pat. No. 5,872,232 to Cook, et al. “2′-O-modified oligonucleotides” and many other references known to one of skill.

In preferred embodiments, PCR by temperature cycling is the amplification method that is used for target nucleic acid amplification. Most of the variations of the PCR are suitable for use with the present invention. However, typically, unsymmetrical PCR wherein PCR primers are used in an uneven ratio or PCR with primers incorporating phosphothioates is a preferred method of choice.

In accordance with certain aspects of the present invention, at least the amplification operation is incorporated into the overall analytical system and is carried out within the microfluidic environment. The devices of the invention optionally provide unique channel geometry whereby a “hot-start” PCR may be carried out by providing a side channel intersecting each of the analysis channels such that one or more reagents may be added to the reaction mixture after the target sequence has undergone annealing so as to avoid formation of primer-primer hybrids. “Hot-Start” PCR has been described in detail in PCT application US01/28646 filed Sep. 13, 2001, which is incorporated by reference in its entirety herein.

In preferred aspects, post amplification detection is also carried out in the same device. Extremely useful methods have been described for rapidly performing PCR amplification in microfluidic channels using electrical energy to heat fluids in which the reactions are carried out. See, e.g., U.S. Pat. No. 5,965,410, which is incorporated herein by reference in its entirety for all purposes.

These electrical heating methods have been incorporated into microfluidic devices that include channel configurations optimized for the performance of that function, and which are described in, e.g., U.S. patent application Ser. No. 60/232,349 filed Sep. 14, 2000, which is incorporated herein by reference in its entirety for all purposes. In order to increase the rate of throughput for the systems and methods of the invention, it is generally desirable to multiplex the sample preparation steps. In preferred aspects, the present invention provides devices whereby multiple target sequences are flowed into a main channel and split into several sample slugs which are directed to several different analysis channels. The primer sequences are introduced into each analysis channel via a well connected to each of the analysis channels. Alternatively, multiplexing is achieved by preparing multiple different target sequences at the same time and preferably, in the same mixture. For example, one can readily amplify multiple different target sequences in a single mixture by including appropriate primer sequences for each target sequence and carrying out the amplification reaction. This leads to multiple amplified target sequences in the same mixture.

Following the sample preparation steps, the target nucleic acid(s) is then subjected to a discrimination step discussed in greater detail, below in which it is determined which polymorphic variant, e.g., SNP, is present at the particular locus or loci of interest. In order to perform the discrimination step, that amplified material is optionally moved to a different region of a microfluidic channel network, e.g., moved out of a amplification region of temperature cycling into a channel or chamber region that is subjected to a temperature gradient for enhancing detection of hybridizations.

B. Discrimination

The main analytical operation of any analysis of polymorphic genetic loci is the discrimination between potential sequence variations at a given sequence location within the sample material. In particular, the target sequence or sequences are analyzed to determine which variant of a particular polymorphism is present at the particular locus or loci in that target sequence. In its simplest form, the discrimination step is merely a confirmation of the presence or absence of a particular sequence at the locus or loci of interest, by identifying the sequence at that locus in the target sequence. In some methods, this step is carried out by sequencing the area of the target sequence surrounding the locus or loci using conventional sequencing technologies, e.g., Sanger sequencing methods. Alternative methods rely upon the activity of an enzyme to distinguish between different sequence alternatives. For example, in primer extension methods, the activity of a DNA polymerase is used to extend an oligonucleotide primer that is to be positioned immediately adjacent to a variant site, e.g., by virtue of the complementarity of the primer sequence to the portion of the target sequence adjacent to the variant position. For example, if primer extension over the variant site is carried out in the presence of differently labeled ddNTPs, one can identify the variant by virtue of the labeled ddNTP that is incorporated at that site. See U.S. Pat. No. 5,888,819 to Knapp et al. incorporated by reference in its entirety herein. If a particular mixture of dNTPs is used, one can identify the next base by examining the extension products using gel electrophoresis, fluorescent based detection or mass spectrometer based methods. Alternatively, enzymes that recognize single base mismatches and cleave them from the primer, e.g., exonucleases, have been used to reveal the identity of the base at the variant position, e.g., by incorporating FRET based dye pairs on the primer/probe, wherein cleavage results in a shift in fluorescent signal, or by size based analysis of the reaction products.

Alternatively, and generally as used in the present invention, the presence or absence of a particular sequence at the locus or loci of interest is determined by hybridizing a complementary (or putatively complimentary) probe to the sequence that includes the locus and detecting whether (and/or to what degree) the hybridization event occurs. Typically, hybridization reactions to determine genetic type or distinguish between sequences that have only small differences, for e.g., 1 mismatch have required a number of different experimental conditions such as temperature, salt concentration etc. in order to be useful. Further, in the past, oligonucleotide probes have typically required sufficient sequence length so that hybridization results could be relied upon with reasonable confidence. In general, as probe length decreases, the stability of the hybrid of the probe and the target sequence diminishes and the more likely sequence variations (e.g., relative proportion of Gs, C, As and Ts) will affect the hybridization reaction. As a result, probes having longer sequences, e.g., 12 or more bases are typically employed. Conversely, as probe lengths increase, differences in stability because of a single base mismatch, e.g., as the result of a particular SNP, are more difficult to detect. Typically, the stability of long oligonucleotide hybrids is analyzed at a series of different temperatures to reliably detect small differences in the target molecule being analyzed. In general, this makes large scale typing difficult to achieve with these methods, given the impracticality of testing large numbers of hybridization reaction mixtures with respect to large numbers of different temperature conditions. However, in at least one aspect the present invention addresses these issues by integrating a thermally controlled detection zone in the device to facilitate the analysis of multiple hybridization at a range of different temperatures, thereby overcoming the problems associated with other systems. In addition, as set forth in more detail herein, preferred embodiments of the invention use short probes which are stabilized by coaxial stacking resulting from hybridizing two or more short probes adjacent to one another on a target nucleic acid.

Other discrimination methods attach one of the probe or the target sequence to a solid support. When the complementary probe (or target) binds to the tethered sequence, and the remaining free probes (or target) is washed away. The bound material is then detected. Examples of these types of methods include conventional spotting and blotting hybridization assays, as well as oligonucleotide array based methods of discrimination, as described above. Another example of sequencing by hybridization uses nucleic acids fixed to beads in hybridization based methods. Examples of this are described in “Manipulation of Microparticles in Microfluidic Systems” WO 00/50172 to Mehta et al. In these methods, beads comprising sequencing reagents are flowed through microfluidic devices, e.g., in sequencing by hybridization methods. WO 00/50172 and related publication WO 00/50642 to Parce et al., “Sequencing by Incorporation,” also describe sequencing by incorporation methods which use reversible chain termination approaches for sequencing by synthesis, e.g., in combination with photobleaching, which provides an alternate approach to sequence detection.

1. Hybridization

In the methods and systems of the present invention, discrimination typically relies upon the hybridization of one or more short oligonucleotides to the sequence region in which the polymorphic locus exists. In particular, short oligonucleotide probes are contacted with the sample DNA, or material derived from DNA, e.g., via DNA amplifications such as PCR, where the probes are complementary to a portion of the sample sequence that includes the polymorphic position, they hybridize with that portion of the target. Where the variant is different, base pairing does not occur. The probe may be complementary to one variant or the other variant at that position. For example, where a particular SNP is a G to T variation in a population, the probe may be complementary to either the sequence portion including either the G or the T. Where it is complementary to one, the ability to hybridize under appropriate conditions indicates the presence of that variant in the sequence portion. The inability specifically to hybridize under given hybridization conditions is then indicative of the other variant. In preferred aspects, analyses involve screening with both variant probes or probe sets, as a positively and negatively controlled experiment.

Conditions for specific hybridization, including for single-nucleotide discrimination are known. Generally speaking, nucleic acids “hybridize” when they associate, e.g., in solution or partially in a solid phase (e.g., when one of the hybridizing nucleic acids is fixed on a solid support). Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel, supra. Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) provide additional details on the synthesis, labeling, detection and quantification of DNA and RNA, including oligonucleotides. Comparative hybridization is a common way of identifying specific nucleic acid interactions. There are many genetic markers that can be detected by hybridization. These include restriction fragment length polymorphisms (RFLPs), allele specific hybridization (ASH), single nucleotide polymorphism (SNP), arbitrary fragment length polymorphisms (AFLP), specific sequence detection (e.g., in sequencing by hybridization or sequence verification by hybridization) and many others.

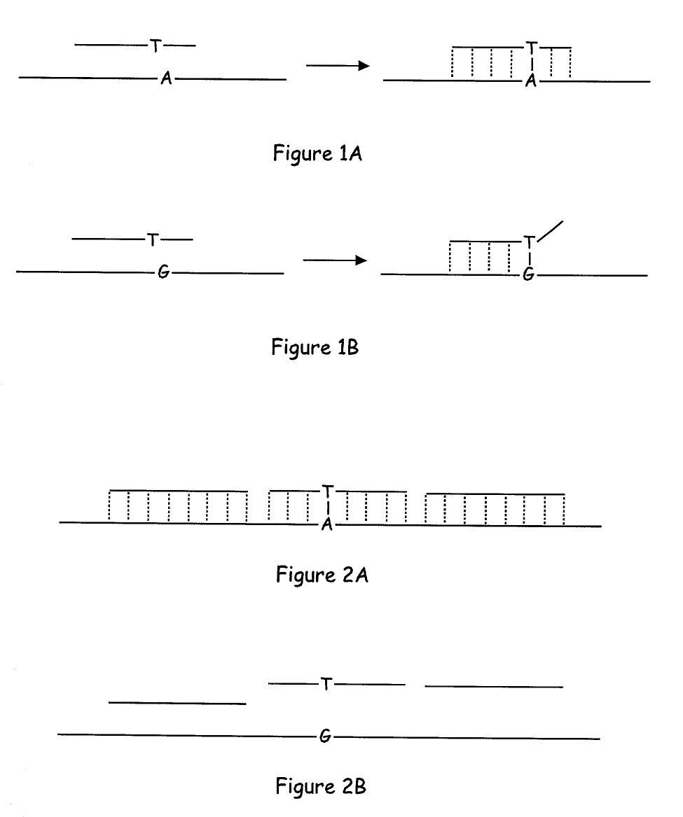

Hybridization based discrimination of polymorphic marker sequences is schematically illustrated in FIG. 1. As shown, a target nucleic acid sequence that includes a known polymorphic locus is contacted with a probe sequence that is complementary to that sequence including at the interrogation position of the probe, e.g., the position that is complimentary to the location of the target that is varied and sought to be identified. If the probe is capable of perfectly hybridizing with the target sequence, then one can identify the base that is present at the locus of interest. If the probe does not perfectly hybridize with the target sequence, then one can make the determination that the base is not the expected base at that position, e.g., it is another variant at that locus. By way of example, and as illustrated in FIG. 1, a target sequence includes a known polymorphic position or locus which may be either an A or a G at the particular locus. The target is interrogated with a first probe that is complementary to the target sequence, including the A variant at the locus of interest (see panel A). In the case where the target includes the A variant, the probe (comprising a T at the interrogation position) hybridizes perfectly to the target and this hybridization is identified (e.g., as described in greater detail below). Where the target includes the G variant, e.g., as shown in Panel B, the probe that is complementary to the A variant does not perfectly hybridize with the target, and this inability is identifiable by the decrease in stability of any resulting hybrid relative to the perfectly matched probe.

In order to enhance confidence in the identification of which variant is present at a given position, the methods described herein often utilize at least two hybridization steps in order to identify the base at a single variant locus (e.g., identify the variant that is present at the locus), also referred to herein as “calling the base.” In particular, probes that are complementary to each known or possible variant at a given position are interrogated separately against the target sequence. This permits positive and negative control of the determination or calling of a variant position. In particular a second probe that is complementary to the target sequence including the known base variation, is contacted with the target sequence, and its ability to hybridize is determined. The extent of hybridization of the two variant probes is then compared and the perfectly matching probe is identified, which in turn, leads to identification of the sequence at the polymorphic locus. In theory, four probes would be necessary to type unambiguously a particular variant nucleotide, each of the probes having a different one of the four nucleotides at the variant site.

a. Universal Library of Short Probes

The present invention provides a unique solution to the problems associated with cost-effective analysis of SNPs and other polymorphic variants. In certain particular embodiments, the methods and systems of the present invention employ libraries of manageable sizes comprising relatively short oligonucleotide probes that can be interrogated against one or multiple sample sequences to identify the particular variants that are present in the sample sequences.

More particularly, the probes used in the methods and systems of the present invention typically employ relatively short interrogation sequences in order to permit the generation of libraries of all or substantially all of the probe sequences possible. By “interrogation sequence” is generally meant the sequence in a probe that is intended to be complementary and hybridize with one variant of a polymorphic locus in a target sequence, as well as the immediately surrounding nucleotides on the 3′ and/or 5′ side of the locus. The term “interrogation base” or “interrogation position” refers to the base or analog within the probe that is intended to interrogate the position of interest in the target sequence, e.g., be positioned adjacent to the position of interest when the remainder of the interrogation sequence has hybridized with the target sequence surrounding or adjacent to the position of interest.

The short nucleic acid probes used in accordance with the present invention typically include an interrogation sequence that is less than or equal to about 10 bases in length, preferably, from about 5 to about 9 bases and more preferably from about 6 to about 8 bases in length. Thus, an overall probe sequence may include substantially more bases, provided that the interrogation sequence portion of the probe is within the parameters described herein. The size of the library of probes is proportional to the length of the interrogation sequences. A library of all possible interrogation sequences of 5 bases in length (5-mers) would include 1024 different probes, while a library of all possible 6-mer probes would include 4096 different probe sequences. By utilizing a library of all possible probe sequences of a given length one can provide a system that can screen for any possible genetic variant using the methods of the invention. Specifically, by having all possible probe sequences, one can select a probe from the library that will hybridize to any possible target sequence segment. In a first aspect, the universal library comprises substantially all possible variations for a sequence of a fixed number of nucleotides. For example, the library will comprise substantially all possible variations of hexamers, amounting to approximately 4096 members. In a preferred embodiment, the library will comprise of 90% of all possible variations. In more preferred embodiments, the library will comprise of at least 95% of all possible variations. In even more preferred embodiments, the library will comprise of 99% to 100% of all possible variations.

In a related aspect, multiple sets of the same library of probes are created whereby member probes of a first set are labeled with first label, members probes of a second set are labeled with a second label, member probes of a third set are labeled with a third label and so on. In a first aspect, a short probe sequence that is complementary to a region that includes a variant locus is stabilized by employing a modular probe stacking technique. By way of example, a standard nucleic acid 6-mer probe will form a relatively unstable hybrid with a complementary target sequence. However, by hybridizing one or more additional probes adjacent to the 6-mer probe, e.g., two 6-mer probes, one on either side of the original probe, one can improve the stability of the hybrid to approximately that of a probe having the combined length of the stacked probes, e.g., three adjacently hybridized 6-mer probes are substantially as stable as a single 18-mer probe.

Further, because the methods and systems of the invention employ a universal library of short modular probes, adding the stacked probes to the hybridization reaction is simply a matter of selecting those probes from the library based upon the known sequence surrounding the portion of the target sequence that is of interest, e.g., the portion including the variant. In accordance with this aspect of the invention, at least two adjacently hybridizing probes are employed, and preferably three adjacently hybridizing probes or more are employed.

Hybrid stability can also be improved by using non-standard “nucleotides” in the probes of interest. For example, PNAs and LNAs both display relatively high stability even for relatively short nucleic acids (e.g., on the order of 5-6 mers). Stability of hybridization can thus be improved for n-mers or adjacent stacked n-mers by using more stable nucleic acids, e.g., which include LNAs or PNAs.

Typically, any of the at least two stacked probes can include the interrogation sequence for the variant position. For example, the probe that is disposed at either the 3′ or 5′ end of the at least two stacked probes may include the variant sequence. Typically, it is preferred that the variant sequence position be located toward the central portion of the combined probes or closer, with a central interrogation position in the interrogation sequence (the base corresponding to the variant position in the target) being most preferred, as single base mismatches within the center of a probe sequence tend to be more destabilizing than those disposed closer to either end.

These stacking methods can also be employed to stack probes from different universal libraries of n-mers. For example, one library would include probes having at their 3′ ends one of two generic complementary sequences, and all possible short oligonucleotide sequences at the 5′ end. These sequences would function as the interrogation sequence for the first library of probes. A second library of probes would include the second of the two generic complementary sequences at the 5′ end and all possible short oligonucleotide sequences at the 3′ end. One would then select a probe from each of the libraries that is complementary to the sequence surrounding a polymorphic locus. Hybridization of these sequences to the target sequence would then indicate the presence of a particular variant at that position. The hybridization reaction would typically be indicated by including one member of a FRET pair on the probes of one library and the other member of a FRET pair on the probes of the other library. The stacked probes are then ligated whereupon they will act as a FRET pair.

FRET (Fluorescence Resonance Energy Transfer) is a non-radiative energy transfer phenomenon in which two fluorophores with overlapping emission and excitation spectra, when in sufficiently close proximity, experience energy transfer by a resonance dipole induced dipole interaction. The phenomenon is commonly used to study the binding of analytes such as nucleic acids, proteins and the like. Additional details regarding FRET are found below.

In operation, at least two or more probes are selected from the multiple sets whereby at least one of the probes is complementary to the target sequence comprising the polymorphic variant and at least one of the other probes is complementary to a region adjacent or substantially adjacent to the region of the first probe such that when hybridized to the target nucleic acid sequence, the two or more probes selected are adjacent or substantially adjacent to each other. In other words, the probe comprising the interrogation base is coaxially stacked with at least one or more probes that form a flanking sequence to the interrogating probe. The multiple probes are thereby coaxially stacked along the target. Therefore, when assembled, the multiple probes form a longer oligonucleotide thereby improving stability of the hybrid. Coaxial stacking of probes allows for improved stability as well as improved specificity depending upon the method of detection employed for determining the hybridization of the probes with the target sequence. These flanking sequences include complementary labeling groups, e.g., members of a FRET pair, a fluorophore and a quencher or the like. In accordance with the present invention, both the interrogation probe and the flanking sequence may be selected from the universal library of short probe sequences. Any combination of anchor and probe sequences may be used with the methods of the present invention. In a preferred method at least three members of the universal library are used to generate an assembled probe comprising at least eighteen bases. The combination of anchors and probes may be an anchor-anchor-probe; an anchor-probe-anchor or a probe-anchor-anchor. Although described as one probe, two anchor combinations, one of skill will appreciate that any number of variations are possible. For example, in certain aspects it may be desirable to use two anchors, two probes; three anchors, two probes or any other similar combination.

In one example, three copies of a library comprising hexamers, for e.g., a 4096 probe library, are designed whereby each copy is labeled with a different label. For example, each set of selected probes may be labeled with two different fluorescent molecules. In that case, FRET, fluorescence resonance energy transfer may be the suitable detection means whereby a fluorescent signal is produced only when both probes are bound next to each other. Fluorescent resonance energy transfer (FRET) is a distance dependent excited state interaction in which emission of one fluorophore is coupled to the excitation of another which is in proximity (close enough for an observable change in emissions to occur). Some excited fluorophores interact to form excimers, which are excited state dimers that exhibit altered emission spectra (e.g., phospholipid analogs with pyrene sn-2 acyl chains); see, Haugland (1996) Handbook of Fluorescent Probes and Research Chemicals Published by Molecular Probes, Inc., Eugene, Oreg. e.g., at chapter 13). The use of different labels and different colored dyes allows for the simultaneous detection of two probe intensities whereby the genotyping of samples is made more robust. For example, in cases of one anchor two interrogation probes sequences, the anchor is labeled with a donor while each of the two interrogation probe sequences is labeled with an acceptor. The donor and the acceptor interact when in proximity. When hybridized to the target sequence, the anchor and the interrogation probe comprising a perfect match with the region of the target comprising the polymorphic variant will be coaxially stacked whereby the proximity of the two labels causes a change in the intrinsic fluorescence of the donor. Many appropriate interactive labels are known. For example, fluorescent labels, dyes, enzymatic labels, and antibody labels are all suitable for use with the present invention. With regard to preferred fluorescent pairs, there are a number of fluorophores which are known to quench one another. Fluorescence quenching is a bimolecular process that reduces the fluorescence quantum yield, typically without changing the fluorescence emission spectrum. Quenching can result from transient excited state interactions, (collisional quenching) or, e.g., from the formation of nonfluorescent ground state species. Self quenching is the quenching of one fluorophore by another; it tends to occur when high concentrations, labeling densities, or proximity of labels occurs Examples of interactive fluorescent label pairs include terbium chelate and TRITC (tetrarhodamine isothiocyanate), europium cryptate and Allophycocyanin, DABCYL and EDANS, Fluorescein and Tetramethylrhodamine, IAEDANS and Fluorescein, Fluorescein and Fluorescein, BODIPY FL and BODIPY FL, and Fluorescein and QSY 7 dye, and many others known to one of skill.

Similarly, other detection means employing suitable colorimetric labels may be used with the present invention. Colorimeteric detection using two calorimetric labels can result in combination which yield a third color, e.g., a blue emission in proximity to a yellow emission provides an observed green emission.

In operation, an interrogation sequence or probe is used in combination with at least one anchor or flanking sequence. The length of the probes and the anchors can be between 5 to 9 nucleotides in length, preferably between 6 to 8 nucleotides and even more preferably, 6 nucleotides in length. Three copies of a library comprising a first group forming variations for the selection of a first probe, a second group comprising variations for the selection of a second probe and a third group with complementary flanking sequences for the selection of anchors. Each probe in the first and second groups is labeled with a fluorescent molecule. Corresponding probes in the first and second groups have different fluorescent molecules and use FRET (fluorescence resonance energy transfer) to produce a fluorescent signal only when both probes are bound next to each other. The group forming the anchors is labeled with a donor molecule of the FRET pair. Each member of the groups comprising the probes are labeled with a different acceptor molecule. Typically, a mixture of one member from each group is assembled with the target sample. The donor is excited and the extent of fluorescence at each of the acceptor molecule wavelengths is monitored to determine the SNP identity.

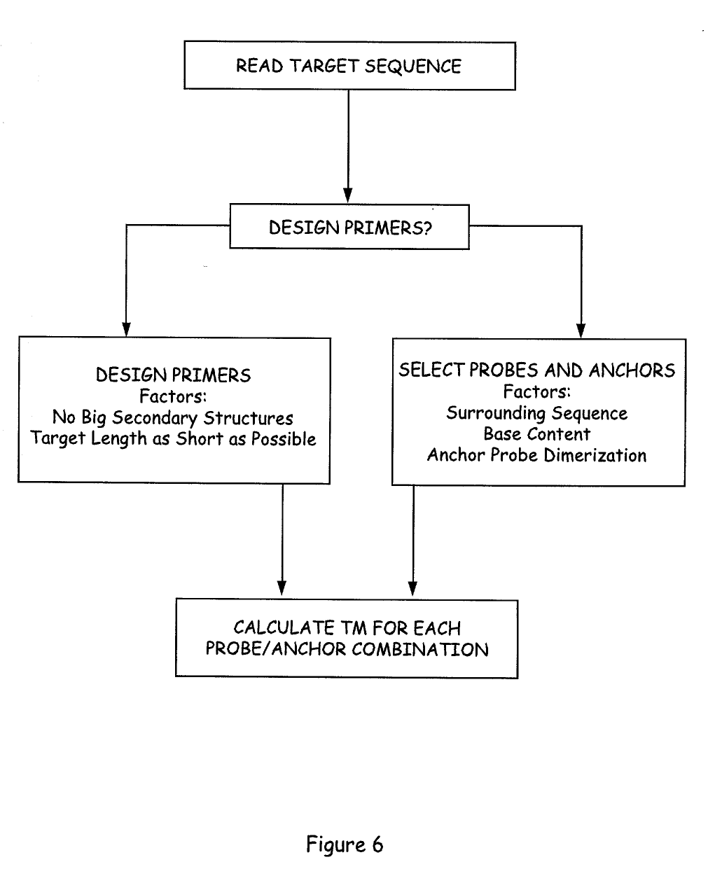

The design of the probe library is typically accomplished using custom software which helps with the prediction of appropriate probe lengths and the selection of suitable probes for a given target sequence. The software algorithms take into account several factors for the selection of the probes for a given target sequence. Typically, the software algorithms consider the base content of the probe sequences for their thermodynamic values, the surrounding sequences of the probes, and the anchor-probe dimerization or cross hybridization to each other. An example flow chart outlining one example embodiment is found in FIG. 6.

One available computer program for primer selection that can be used or adapted to the present invention is the MacVector™ program from Kodak. An alternate program is the MFOLD program (Genetics Computer Group, Madison Wis.) which predicts secondary structure of, e.g., single-stranded nucleic acids. In addition to programs for primer selection, one of skill can easily design simple programs which select for any hybridization or probe interaction criteria of interest.

In further advantage over previous methods and systems, the present invention also provides a universal library of probes within a readily accessible storage format, e.g., readily accessible to the reaction vessel for the discrimination operation, e.g., a microfluidic channel network. Alternatively, more conventional probe storage systems may be employed, including higher capacity multiwell plates, e.g., 96, 384 or 1536 well plates, which maintain the different probes in separate fluid wells. For ease of storage, reagents can also be stored dry in a micro titer plate. Only a relatively small number of plates would generally be employed to store the entire library of short probes used in conjunction with the present invention. By way of example, the universal probe library is optionally provided in an immobilized form, e.g., spotted and/or dried, in different, known locations on a planar substrate or card. The system is then capable of sampling the probes from the card and introducing them into the reaction channel or chamber of a microfluidic device. Examples of such LibraryCard™ systems are described in U.S. Pat. No. 6,042,709 and U.S. patent application Ser. No. 09/750,450, filed Dec. 28, 2000, each of which is incorporated herein by reference in its entirety for all purposes. A variety of systems comprising sources of materials and the interface between such sources and microfluidic devices are also set forth, e.g., in Knapp et al., “Closed Loop Biochemical Analyzers” (WO 98/45481; U.S. Pat. No. 6,235,471); in U.S. Pat. No. 5,942,443 entitled “High Throughput Screening Assay Systems in Microscale Fluidic Devices,” issued Aug. 24, 1999 to J. Wallace Parce et al.; in WO 00/50172 “Manipulation of Microparticles in Microfluidic Systems,” to Mehta et al.; and, e.g., in WO 00/05642 to Parce et al. “Sequencing by Incorporation.”

In some cases, it is desirable to incorporate longer probes in the universal library for those sequences that are known to be less stable, e.g., those sequences made up substantially or entirely from A and T bases. Accordingly, the universal libraries described herein optionally include probes of a first length, e.g., 6-mers for all sequences except those including a substantially or entirely A and/or T sequence within the 6 base interrogation sequence. A library of all possible 6-mers has the size of 4⁶ which therefore has 4096 members. For the probes that include the 6 base interrogation sequence that is made up of A and/or T bases, an additional base or bases is added to the probe to correspond to all possible 1, 2, 3 or 4 base extensions of those probes. For example, all possible 1-mer extensions of the AT probes described above would be included in only 256 additional different probes to a library of all possible 6-mers, if extended in one direction, twice that (e.g., 512) if extensions were desired in the both directions. Similarly, addition of all possible 2-mer extensions to one end of the 100% AT 6-mer probes would add 1024 additional probes to the size of the library, twice that if extensions are made in the other direction, or three times that if the extensions are one base in each direction. Thus, in preferred aspects, the probes including all A and/or T bases include extensions of 1, 2, 3 or 4 bases and include all possible 1, 2, 3, or 4 base extensions of all possible entirely A and/or T 6-mers. Accordingly, a universal probe library of all possible 6-mers and all possible two base extensions (off either end or off both ends) of the A and/or T 6-mers would include 7168 different members, each of which would be reversibly immobilized on a solid support, e.g., a LibraryCard™ reagent array or disposed in a separate well of a number of multiwell plates, e.g., 19 384-well plates, or 5 1536-well plates.

Because the universal library of probes includes all possible probe sequences of a given length, e.g., less than 10 bases and preferably from about 5 to about 8 bases, and because target nucleic acid sequences of interest are typically present in relatively non-complex mixtures of sequences (e.g., PCR products that are substantially comprised of the target sequence with low levels of other, possibly cross reacting sequences), these probes can be used to hybridize to any possible nucleic acid sequence in a target sequence. In addition, these universal probe libraries can also be used to build longer probes, by hybridizing the various shorter probe pieces to adjacent portions of the target sequence. These pieced together longer probes are then optionally ligated using, e.g., DNA ligase enzymes (i.e., T4 DNA ligase), producing a single longer probe sequence that may be used in either hybridization experiments or as a primer for amplification reactions (changing the size of the probes can also be detected, e.g., using FP, or electrophoresis, providing an additional detection modality).

For example, where one desires a probe that is complementary to a stretch of 18 contiguous bases in a target sequence, one can select the three 6-mer probes that, when placed end to end, complement that sequence. This adjacent hybridization reaction is facilitated by the enhanced thermodynamic stability imparted to adjacently hybridized probes, also referred to as “stacking forces.” These stacked probes effectively function as a single 18 base probe, and they can be ligated to form an 18 base probe which can be detected or used subsequently. Use of short, stacked probes to build larger probing sequences (for use in primer walking sequencing methods) was described, e.g., in Published PCT Application No. WO 98/45481, and U.S. Pat. No. 5,547,843 to Studier et al., each of which is incorporated herein by reference in its entirety for all purposes. See also, Knapp et al., “Closed Loop Biochemical Analyzers” (WO 98/45481; U.S. Pat. No. 6,235,471).

FIG. 2 schematically illustrates the use of stacked, optionally ligated probes as a means for obtaining higher specificity in the hybridization reactions of the invention. In particular, three short probes are hybridized to the target sequence at the variant locus. In the case of perfect hybridization, all probes perfectly hybridize, imparting greater stability on the aggregate probes. In the case of a non-perfect match at the variant position, e.g., by one probe such as the central probe, that probe is destabilized, thereby destabilizing the aggregate of probes to a greater degree than would be the case using a single long probe.

b) Probe Types

The probe types used in conjunction with the present invention can vary. In particular, the probes used in the hybridization/discrimination steps of the methods of the present invention include DNA probes, RNA probes, and nucleic acid analog probes, such as peptide nucleic acids (PNAs) or locked nucleic acids (LNAs), or mixtures of any of these types of materials, e.g., LNA/DNA probes, in the interrogation sequence or employing one type of probe as the interrogation sequence and another type of material in the connected ancillary sequences (within the overall probe but outside of the interrogation sequence).

As noted above, nucleic acid probes, e.g., DNA probes, are readily employed in the discrimination portion of the methods and systems of the present invention. Synthesis and use of DNA probes in hybridization reactions has been well studied. Alternatively, and in many preferred aspects, nucleic acid analogs or mixtures of nucleic acids and nucleic acid analogs are employed as probes in accordance with the present invention.

One type of particularly preferred class of nucleic acid analogs is peptide nucleic acids (PNAs) which generally comprise a peptide backbone with nucleobase-like side groups, which are capable of hybridizing with complementary target nucleic acids (see, See European Patent applications EP 92/01219 and 92/01220). PNA bases provide advantages over pure nucleic acid bases in a number of respects. For example, PNA sequences are uncharged. Accordingly, one can monitor their hybridization to a target sequence by detecting a change in the charge of the labeled hybrid over the unbound labeled PNA probe. Methods of effecting such detection are described in detail in U.S. patent application Ser. No. 09/569,193, filed May 11, 2000, and incorporated herein by reference in its entirety for all purposes. Briefly, a labeled PNA probe is contacted with a target nucleic acid sequence. In the event the probe hybridizes with the target, the target, by virtue of its highly negatively charged nucleic acid backbone, imparts a substantial negative charge to the otherwise uncharged probe. The hybrid is then exposed to a large, oppositely charged molecule, e.g., polylysine, or polyarginine, which imparts a substantial mass change upon the labeled hybrid by virtue of the charge. This mass change is then readily detectable using, e.g., fluorescence polarization based detection. In the event the probe does not bind, the labeled probe lacks any charge to give rise to a mass change, and thus is not detected. PNA probes also include higher affinity than DNA probes, increased specificity. Alternatively, the electrophoretic mobility of the PNA probe can be measured using a variety of conventional methods such as slab gel or capillary electrophoresis. The labeled PNA probe will not migrate towards the anode in its free form at the same rate that it will when hybridized to a target molecule of DNA. PNAs also offer advantages in that the peptide backbone offers greater flexibility in creating novel modifications, adding functional groups that facilitate assays,

In alternative preferred aspects, the probes employed in accordance with the present invention comprise locked nucleic acids (LNAs) within the interrogation sequence. Locked nucleic acids have the same phosphodiester backbone as naturally occurring nucleic acids, but have a different conformation in the sugar moiety, e.g., bi- or tricyclic structure. LNAs provide advantages over naturally occurring nucleic acids because of their greater hybridization stability. This allows the generation of much shorter probes which are destabilized more dramatically by single base mismatches with the target as compared to perfectly matched probes, giving rise to easier discrimination of the base present at the polymorphic locus (see, International Patent Application No. WO 99/14226, which is incorporated herein by reference in its entirety for all purposes).

In yet another aspect, the probes employed comprise of an oligonucleotide sequence linked to a minor groove binder (MGB). Typically, the oligonucleotides have a plurality of nucleotide units, a 3′end and a 5′end, and a minor groove binder moiety covalently attached to at least one of the nucleotides. A minor groove binder moiety is a radical of a molecule having a molecular weight of approximately 150 to approximately 2000 daltons. The oligonucleotide linked to the MGB may include phosphorothiotes and methylphosphonates in addition to the “natural” phosphodiester linkages. An oligonucleotide-minor groove binder (ODN-MGB) conjugate provides advantages over naturally occurring nucleic acids because while the oligonucleotide portion of the molecule binds to a complimentary sequence in single stranded DNA, RNA, double stranded DNA, and DNA-RNA hybrid, the MGB is incorporated in the newly formed “duplex” and thereby strengthens the bond increasing the hybridization stability. MGBs are described in detail in U.S. Pat. No. 6,084,102 which is incorporated by reference in its entirety herein.

In a related aspect, the oligonucleotide of the ODN-MGB conjugate may also have a relative low molecular weight “tail moiety” attached to either the 3′ or the 5′ ends or both. The tail moiety is different from the MGB moiety and is attached to the end of the oligonucleotide which does not have the MGB. The tail moiety may be a phosphate, a phosphate ester, an alkyl group, an aminoalkyl group, or a lipophilic group.

The MGB oligonucleotide conjugates typically have a preference for AT rich regions of double stranded DNA. However, modification of the conjugate allows for the design of MGB-ODN conjugate which have a preference for C-G rich regions. For example, replacing the guanine with hypoxanthine is one such modification.

In another aspect, the probes may comprise an oligonucleotide wherein at least one of the bases is a modified base. These probes are termed “modified base probes.” Short probes comprising modified bases can be used for hybridization detection because the modified bases offer more versatility in the design of probes for a universal library. Modified bases are commercially available from several oligonucleotide manufacturers including Synthetic Genetics, 3457 Industrial Ct., San Diego, Calif. 92121, SIGMA Genosys, 1442 Lake Front Circle, The Woodlands, Tex. 77380. One example of a modified base is a probe comprising a modified G base available from Synthetic Genetics wherein the G is replaced with a PPG allowing for the design of probes containing more than four Gs in a row.

Regardless of the specific structure of the interrogation sequence, e.g., DNA, PNA, LNA, ODN-MGB, modified bases, or mixture of any or all of these, the probes also typically employ a detectable property that permits their detection and even more preferably, permits or at least does not foreclose the possibility of their detection and differentiation in free and hybridized forms.

In the case of hybridization detection methods that rely upon a detection in the change of the charge and/or mass of the hybrid, e.g., fluorescence polarization, incorporation of a detectable label is simply a matter of including a detectable moiety, e.g., a fluorescein or rhodamine based fluorescent dye, on the probe sequences. This dye is then detected and attributed to either a free or a bound probe by virtue of the mass and/or charge change.

Preferably, however, probes are employed that generate or quench a fluorescent signal upon the occurrence of the hybridization event, e.g., either produce or quench fluorescence. Particularly preferred examples of a fluorogenic probes include self-hybridizing probes that form a hairpin loop structure, and include complementary labeling groups that are either quenched or fluorescent when in the hairpin loop structure. Briefly, such probes comprise a nucleic acid sequence that includes an interrogation sequence in its central portion, which is flanked on either side by two complementary nucleic acid sequences. At each flanking end is disposed one member of a label pair, e.g., a FRET pair or a donor-quencher pair. In the free state, e.g., unhybridized to a separate target sequence, the flanking regions hybridize to each other forming the stem to the hairpin loop structure in which the interrogation sequence forms a single stranded loop at the top of a double stranded stem. The labeling pair is positioned in relatively close proximity to each other when the overall probe is in the hairpin conformation. The close proximity results in generation or quenching of a fluorescent signal from the labeling pair, depending upon the nature of the pair. In the presence of a target nucleic acid sequence that is perfectly complementary to the interrogation sequence, the thermodynamic stability of the hairpin loop structure is lower than the stability of the hybrid of the interrogation sequence and the target sequence. This results in hybridization of the interrogation sequence to the complementary portion of the target sequence. This results in the complementary flanking regions of the probe being prevented from hybridizing to one another, which keeps the label pair sufficiently far apart to either allow an unquenched fluorescent signal or failure to generate a signal which requires both members of the labeling pair. Such probes are similar to the Molecular Beacons described by Kramer et al. in U.S. Pat. No. 5,925,517, except that they are shorter in the length of the interrogation sequence, and in preferred aspects, are mixtures of nucleic acids and nucleic acid analogs.

By way of example, in the case of a 6-mer probe or probe library, a hairpin loop probe that includes a central 6-mer interrogation sequence is employed which is flanked by two complementary sequences. The probe may be entirely made up of naturally occurring nucleic acids, e.g., DNA, or it may be made entirely of nucleic acid analogs, e.g., LNA or PNA, or it may comprise a mixture of DNA and analogs. For example, in at least one particularly preferred aspect, the probes employ an LNA interrogation sequence with DNA flanking regions. In particularly preferred aspects, the interrogation sequence comprises LNA bases while the flanking regions are selected from sequences of DNA and LNA bases. Use of LNA interrogation sequences in the hairpin loop probes results in a substantial improvement over pure nucleic acid probes, in terms of sensitivity for single base mismatch discrimination, as well as higher thermodynamic stability of shorter probe hybrids, e.g., 10 bases or less in the interrogation sequence.

Other analogs may be incorporated into the interrogation sequence to, e.g., stabilize otherwise unstable hybrids, e.g., AT rich sequences. For example, in place of the A base (or A base analog) in the interrogation, one can employ an analog that forms more stable hydrogen bonds with the complementary T base, e.g., 3 hydrogen bonds instead of two. One example of such an analog is 2-amino adenosine, which forms three hydrogen bonds with the Thymidine base.

Universal libraries of these hairpin loop probes are readily assembled using stem sequences of a variety of different potential lengths and sequences. For example, the complementary flanking or stem sequences need to be only of sufficient length to be able to hybridize to each other with a thermodynamic stability that is comparable to or better than that of the single base mismatch hybrid of the interrogation sequence and the target sequence of interest. Typically, such flanking regions are at least 4 bases in length, and preferably, 4, 5, or 6 bases in length. These flanking regions may be common throughout the universal library of probes, e.g., all probes will include the same flanking sequences, or they may be varied throughout the library, in order to avoid interactions between the flanking sequences and the target sequence, e.g., where the interrogation sequence is similar or identical to one of the flanking sequences, or varied to provide appropriate relative stability as compared to the interrogation sequence and target. For example, in preferred aspects, the flanking sequences will be selected so as to be sufficiently stable to form the hairpin loop structure, but not so stable so as to prevent hybridization of the interrogation sequence to the target. For example, in the case of AT rich interrogation sequences, it will generally be desirable to provide flanking regions that are also AT rich. Conversely, where the interrogation sequence is GC rich, it will generally be desirable to provide a more stable (or GC rich flanking sequence) in order to provide the hairpin structure with more stability than a single base mismatch, but not as much stability as the perfectly matched interrogation sequence/target hybrid.

Typically, the flanking sequences comprise more AT rich sequences, e.g., as compared to the interrogation sequence, that are shorter than the interrogation sequences (due to their lower thermodynamic stability and reduced ability to hybridize to the target sequence). In particular, as noted above, in the case of the AT rich interrogation sequences, it is typically desirable to provide extensions of these sequences, resulting in longer interrogation sequences, e.g., 7, 8, 9 or 10 bases in length. As a result, the stems would be shorter, being in the range of 4, 5 or 6 bases in length.

c) Stringency

As noted above, shorter probes form less stable hybrids with target sequences. As a result, under any given set of hybridization conditions, it is more difficult to rely upon differential hybridization of two probes, one of which is complementary to one variant at a given position while the other is complementary to the other variant at that position. In particular, because shorter probes are less stable, it is more difficult to attribute a destabilized hybrid to a single base mismatch than simply to a reduced stability of a shorter probe. As a result, it is more difficult to detect a difference in hybridization of a short, single base mismatched probe to a target sequence and a perfectly matched short probe to that target sequence. This difficulty is further amplified by sequence dependent differences in hybrid stability, e.g.; GC versus AT rich probe sequences (See, e.g., Tijssen (1993), Ausubel, Hames and Higgins 1, and Hames and Higgins 2, all above). In accordance with the present invention, a number of different steps may be employed to either stabilize the hybrid formation, or identify perfectly matched hybrids despite this instability, such as the use of coaxial stacking of adjacent bases, as described throughout.

Because the methods and systems of the invention can employ microfluidic channel networks, varying of the applied temperatures for hybridization reactions can be carried out extremely rapidly, accurately and automatically. For example, in preferred aspects advanced temperature control methods are applied to microscale channels in which hybridization reactions are being carried out to perform the temperature sweep for those reactions. Generally, these methods employ the direct electrical heating (also termed “Joule heating”) of fluids within microscale channels to carry out the temperature variation. Further, by monitoring the electrical parameters of the fluid, e.g., conductivity, one has a built-in temperature monitoring and regulation mechanism. This temperature control method, as well as devices and systems for carrying out this method, are described in substantial detail in U.S. Pat. No. 5,965,410, which is incorporated herein by reference in its entirety for all purposes.

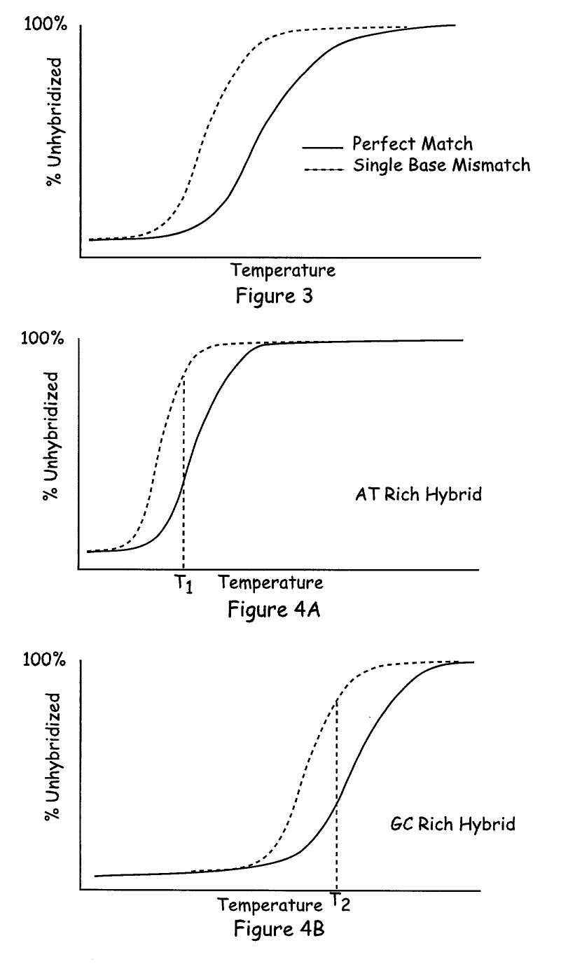

The temperature profile for two hybrids for a variant target sequence provide more readily ascertainable differences than detection of differential hybridization at a single temperature. This is illustrated in FIGS. 3. Briefly, FIG. 3 illustrates the detected signal that is indicative of hybridization of a probe to the target, from hybridization reactions between a single base mismatch probe (dotted line) to a target sequence and a perfectly complementary probe (solid line) to the target sequence. For example, the illustrated signal is optionally a fluorescent signal from the hybridization of molecular beacon probes to the target sequence. Molecular beacon probes typically include a short interrogation sequence that is flanked on either side by two, complementary sequences that include one member of a FRET or fluorescent quencher donor pair as the labeling moiety. In the absence of hybridization to a target sequence by the interrogation sequence, the flanking regions hybridize, bringing the labeling pair in sufficient proximity to each other to quench or emit a fluorescent signal.

Hybridizations at a single temperature can yield only a limited amount of information regarding the hybridization to a target sequence of single base mismatch and perfectly complementary probes having relatively short lengths. In particular, the temperature at which one can optimally discriminate between hybridization of a target to a perfectly matching probe and single base mismatch probe is highly sequence dependent. For example, AT rich probes are generally destabilized or melted at lower temperatures. As a result, optimal discrimination between perfect and imperfect hybrids is generally accomplished at lower temperatures. The converse is true for GC rich probes which have higher melting temperatures.

As such, it is highly unlikely that a single set of reaction conditions could be used that would provide adequate discrimination between perfectly matched and single base mismatched probes for any reasonable number of different sequences.

By obtaining a temperature profile for each hybridization reaction, one can more readily identify which probe perfectly hybridizes with the target sequence. Further, the shape of the melting curve for a single base mismatched probe, or more particularly, the comparison of the shape of that melting curve to a melting curve for a perfectly matched probe and target sequence is far less dependent upon the particular sequence that is being interrogated. As a result, one can use the thermal sweep method in conjunction with libraries of much shorter probes in the hybridization based discrimination methods described herein, and can use them across the spectrum of different sequences that are to be interrogated/hybridized. Using the thermal sweep methods described herein, one can effectively employ shorter probe lengths, can effectively discriminate single base mismatches from perfect matches across all possible probe sequences, and can still obtain the requisite confidence in the results of those hybridization reactions.

FIG. 4 schematically illustrates thermal sweep based detection of hybridization reactions for two representative sequence types. In particular, the graph illustrates a fluorescent signal versus temperature where the fluorescent signal is indicative of strand separation or melting. Such a signal is representative of, for example, a molecular beacon probe hybridized to a target sequence and melted off under increasing temperature. The first plot in FIG. 4A illustrates the melting profile of a perfectly matched probe (solid line) and single base mismatch probe (dotted line) for an AT rich probe/target sequence. The second plot in FIG. 4B illustrates the same signal for a less AT rich, e.g., a GC rich probe/target hybrid for a perfect match (solid line) and a single base mismatch (dotted line). As can be seen, the temperature at which one can optimally discriminate between perfectly matched and imperfectly matched hybrids differs considerably for the different sequences. Further, in some cases, the optimal temperature for one sequence may be totally ineffective as a discrimination temperature for another. Despite this, however, use of a thermal sweep for discrimination allows one to traverse the conditions that are optimally effective for the given discrimination reaction.

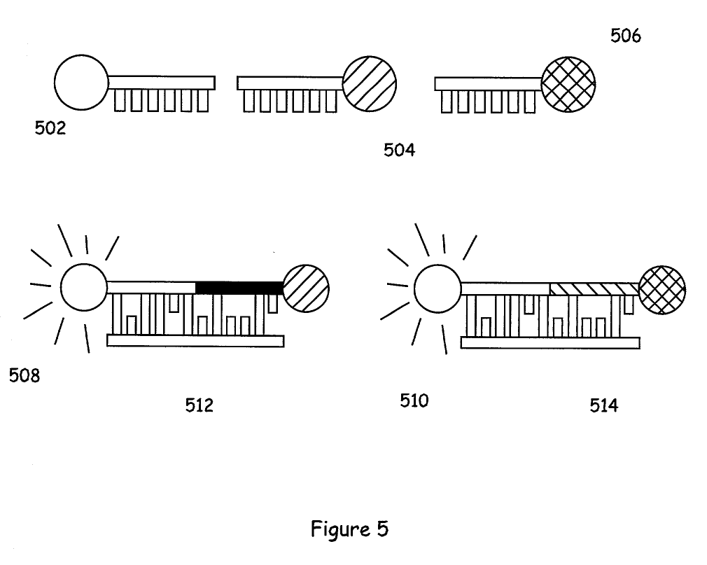

Briefly, one can monitor a hybridization reaction while one varies the temperature of the reaction mixture through the melting point of the hybrid. Where a single base mismatch is present in the hybrid, it will result in a lower melting temperature for the hybrid than in the situation where there is a perfect match hybrid. This difference in melting temperature is far more reliable for shorter probes than simply examining differential hybridization of those shorter probes at a single temperature. In addition, the respective Tm of probes having the same length but differing in sequence can be dramatically different. FIGS. 5A and 5B illustrate the melting temperatures of matched and mismatched hybridizations for two different target sequences using probes labeled with FAM and VIC.

Alternatively, or additionally, the hybridization reaction can be assured/monitored using more complex detection methods, such as 2D-FIDA methods developed by Evotec, Inc. Briefly, 2D-FIDA is one of several single molecule fluorescence detection methods that have recently been optimized for use with a variety of biochemical assays. An instrument system having confocal fluorescence optics allows the optical interrogation of a 1 μm³ (1 femtoliter). By taking very rapid data measurements, the properties of single fluorescent molecules can be observed. Software algorithms can analyze the data and correlate the very brief changes in fluorescence with properties such as diffusion rate, polarization state, fluorescence intensity, etc. These data can, in turn, indicate the level of hybridization by monitoring the frequency at which pairs of probes are detected versus individual probes.

2. Detection

As alluded to above, there are a number of different methods that can be employed in detecting whether or not a particular probe sequence is capable of hybridizing to a particular locus on a target sequence, in accordance with the present invention. These include the use of separation based methods (e.g., hybridization of labeled probes to a target sequence, either free or tethered, followed by separation of the free probe from the hybridized probes), fluorogenic methods (e.g., methods that produce a change in fluorescent emissions from the reaction mix as a result of the hybridization reaction) fluorescent polarization based methods (e.g., methods that rely upon the change in size and/or charge of the hybrid, as compared to the probe, either alone, or with the addition of other reagents), and methods that require further manipulations in order to produce a signal that is indicative of hybridization (e.g., methods that require a transcription or replication operation to generate a signal from the hybrid). These methods are described in greater detail, below.

a) Separation Based Methods

In a first, simple format, the hybridization reaction between a probe and a target system in accordance with the present invention, may be detected by separating labeled free probe from the labeled probes that are hybridized to a target sequence. Such separation can be accomplished as a result of immobilization of the hybrid to a solid phase or it can be based upon a differential mobility of the free probe and the hybrid, e.g., in an electrophoretic separation. In the case of solid phase methods, the target sequence is optionally tethered to a solid support, e.g., a surface of a microfluidic channel or chamber, or another solid support, e.g., beads or resins, disposed in a microfluidic channel or chamber. The probe is then passed over the immobilized target and free probe is washed away. The solid phase is then interrogated for the presence of the labeled probe.

In the case of microfluidic systems incorporating separation-based detection, it is typically preferable to perform the hybridization reaction entirely in a liquid phase. Following the hybridization reaction, the hybrid and any unbound probe are subjected to a separation operation, which preferably involves injection of the mixture from a mixing channel or chamber, into a separation channel, followed by electrophoretic separation of the hybrid and the probe. Because the hybrid, including the target sequence, will be substantially larger than the probe, alone, it will move more slowly through an electrophoretic separation matrix. Microfluidic systems for use in electrophoretic separations of nucleic acids and the like include, e.g., the Agilent Technologies 2100 Bioanalyzer, and associated Caliper LabChip® devices and reagents. Such systems are described, for example, in U.S. Pat. Nos. 6,042,710 and 6,153,073, each of which is incorporated herein by reference in its entirety for all purposes.

b) Fluorogenic Methods

While separations based assays are useful in quantifying hybridization reactions, it is generally preferred to use detection methods that employ fewer operations. In particularly preferred aspects, fluorogenic methods are used to monitor the hybridization reactions that are used to discriminate which variant is present at the polymorphic locus. As used herein, a fluorogenic hybridization detection method is a method that generates a change in the quantity of a fluorescent signal, either an increase or decrease, as a direct or indirect result of the hybridization reaction.

By virtue of the complementary labeling groups, the probe will either yield a fluorescent signal or quench a fluorescent signal upon hybridization to the target sequence. This change in fluorescent signal is then monitored using conventional fluorescent detectors, e.g., epifluorescent microscopes equipped with light detection systems, e.g., photomultiplier tubes, photodiodes, imaging systems, e.g., CCDs, or the like.

In one embodiment, detection of the hybridization reaction can be accomplished using other methods where the hybridization reaction indirectly changes the amount of a fluorescent signal. In a first example of such methods, the short oligonucleotide probe incorporates a labeling moiety that is selectively cleaved off of the probe only when that probe is hybridized to the target sequence. Examples of such probes include, e.g., Taqman™ probes that are generally commercially available from a variety of sources, including, e.g., PE Applied Biosystems (Foster City, Calif.). Briefly, the short oligonucleotide probes of the invention include a labeling group at one terminus. Once the probe is hybridized to the target sequence, the hybrid is mixed with a polymerase enzyme that has exonuclease activity, in the presence of the various dNTPs and a primer sequence that is upstream (e.g., in the 3′ direction) of the locus of interest. The polymerase extends the primer sequence, displacing the probe, cleaving and releasing the 3′ coupled labeling group, which then produces a fluorescent signal.

Another example of probe compositions that can indirectly yield a fluorescent signal following the hybridization reaction involves the use of a two probe system, where the first probe comprises an interrogation sequence, e.g., is complementary to the locus of interest, and the second displacement or invader probe that is complementary to an adjacent portion of the target sequence, and overlaps in complementarity with at least one base of the overall probe sequence, e.g., the interrogation sequence or a flanking sequence. The overlapping nature of the invader probe results in the generation of a flap base or bases of the interrogation probe that is unhybridized to the target sequence (in at least some instances). Treatment of the overall two probe-target hybrid with an appropriate 5′ exonuclease, e.g., Taq polymerase, FEN, etc., results in cleavage of the flap of the interrogation probe sequence, which produces a fluorescent signal, e.g., as the result of a change in relative position of a FRET pair.

In accordance with the present invention, both the interrogation probe and the displacement/invader probe may be selected from the universal library of short probe sequences.

In a related aspect, other labels that are selectively cleaved or otherwise separated from only the hybridized probes are used. Such probes and labels may include labeling groups that are distinguishable from each other on the basis of emission spectra, molecular weight, net charge, affinity, or the like, to allow multiplexed reactions to be carried out, and their results to be differentially determined.

c) Fluorescence Polarization Based Methods

In another aspect, the hybridization reaction can be detected by virtue of a change in size of the labeled probe when it is hybridized to the much larger target nucleic acid sequence, e.g., by fluorescence polarization spectroscopy. In brief, when a fluorescent compound is excited with a polarized light source, it will emit a polarized fluorescence. In the case of small fluorescently labeled molecules in solution, the rotational diffusion rate (spinning and tumbling) of the molecule is relatively fast, which results in a depolarization of the emitted fluorescence in response to a polarized excitation light. As the size of labeled molecules increase, it decreases their rotational diffusion rate, e.g., they rotate, spin and tumble more slowly which results in a more polarized fluorescent emission.

In the case of hybridization reactions, a relatively small, labeled oligonucleotide probe has a first rotational diffusion rate that is relatively fast, due to the small size of the probe. This first rotational diffusion rate results in a certain level of depolarization of fluorescence when excited with a polarized light source. When this probe hybridizes to the target nucleic acid, the labeled hybrid (by virtue of the label on the probe) then has a substantially reduced rotational diffusion rate, due to the increase in size of the hybrid over the probe from the addition of the target sequence. The hybrid depolarizes the fluorescent emissions to a much smaller extent, resulting in much higher level of polarized fluorescence emitted from the hybrid. This change in the level of depolarization is monitored and used to indicate whether the hybridization reaction has occurred. Examples of fluorescence polarization detection systems are generally described and illustrated in, e.g., Published PCT Application NO. WO 99/64840, which is incorporated herein by reference in its entirety for all purposes.

In the case of probes that comprise uncharged nucleic acid analogs, e.g., PNA probes, a modified polarization detection method is optionally employed that yields more robust results than more conventional polarization methods, e.g., as described above. Briefly, and as alluded to previously, a labeled (and uncharged) PNA probe is contacted with the target sequence. Where a hybrid is formed between the uncharged probe and the highly charged target sequence, it will result in a substantial increase in the charge associated with the label, e.g., the charged hybrid versus the uncharged probe. The charged hybrid is then contacted with a relatively large, oppositely charged polyion, e.g., polyarginine, polylysine, etc., which associates with the charged hybrid by virtue of the opposite charge. This results in a substantial increase in size of the labeled hybrid over the labeled free probe, and the consequent decrease in rotational diffusion rate, which is then monitored using fluorescence polarization spectroscopy. Where the probe does not hybridize to the target, it will remain uncharged, and will thus, not associate with the highly charged polyion, and will accordingly not have an altered rotational diffusion rate. This latter method is particularly useful where the target nucleic acid sequence is not sufficiently large to impart an easily detectable change in the rotational diffusion rate of the hybrid over the free probe.

d) Fluorescent Resonance Energy Transfer

In another aspect, detection of the hybridized probes may be accomplished by Fluorescent Resonance Energy Transfer (FRET) (Wttwer, C. et al. Biotechniques 22:130-138, 1997; Bernard, P. et al. Am. J. Pathol. 153:1055-1061, 1998). This method relies on the independent binding of labeled DNA probes on the target sequence. The proximity of the two probes when they are bound to the target sequence affects the increased resonance energy transfer from one probe to the other, leading to a unique fluorescence signal. In embodiments using an anchor and probe combination of short oligonucleotide sequences, the anchor is labeled with a FRET donor dye. Each of the probes is labeled with an acceptor dye. The hybridization of each of the probes with the target sequence is detected by the quenching of the fluorescence of the donor. Examples of interactive fluorescent label pairs include terbium chelate and TRITC (tetrarhodamine isothiocyanate), europium cryptate and Allophycocyanin, DABCYL and EDANS, Fluorescein and Tetramethylrhodamine, IAEDANS and Fluorescein, Fluorescein and Fluorescein, BODIPY FL and BODIPY FL, and Fluorescein and QSY 7 dye. Typically, the acceptor and donor dyes are different. Optionally, the donor and at least one of the acceptor dyes may be the same. In that case, hybridization can be detected by fluorescence polarization, or by any applicable fluorescent signal detection strategy.

e) Multi-label Detection Strategies and Multiplexing

In order to multiplex hybridization reactions, one can employ probes that include labeling groups that have differing emission spectra. These differently labeled probes may be as to different variants at a given position (allowing simultaneous comparison of perfectly matched and single base mismatched probes at a given locus) and/or they may correspond to different polymorphic loci in the same or different target sequences that are all present in the same reaction mixture (allowing multiplexed analysis of multiple loci in one or multiple target sequences). Briefly, creation of a signal of a particular emission spectrum will be indicative of a particular hybridization event, e.g., hybridization to one variant of a particular locus. This multiplexing of hybridization reactions is optionally increased by employing detection strategies that employ different emission spectra for different loci, while employing other detection strategies for distinguishing one variant from another, e.g., fluorescence polarization detection using two probes having the same labeling group.

By way of example, a target sequence that includes two different polymorphic loci is probed with four probes. The first two probes are labeled with a first labeling group and are complementary to the two variants at the first locus. The second two probes are labeled with a second fluorescent group having a different emission spectrum from he first two probes, and are complementary to the two variants at the second locus. Using fluorescence polarization detection with two-color optics, e.g., separately detecting each emission spectrum, one can simultaneously determine which variant is present at each locus.

In a variation of the basic hybridization reactions described herein, and in accordance with the present invention, a two-dimensional detection strategy is optionally employed. Briefly, this detection strategy simultaneously exposes the target sequence to three differently labeled probe sequences. The first probe is specific to the target sequence in a region other than the locus of interest, and is labeled with a fluorescent label having a first emission spectrum. The second probe includes an interrogation sequence that is complementary to one variant of the locus of interest and is labeled with a label having a second emission spectrum that is different from the first. The third probe includes an interrogation sequence that is complementary to the other variant of the locus of interest, and is labeled with a labeling moiety that has a third emission spectrum that is different from both of the first and second emission spectra. As a result of these differing labels, one can simultaneously detect the amount of target and the particular variant that the target includes at the locus of interest, using appropriate filtering optics in the detection system. In a further variation, the third probe could be labeled with a labeling group that has the same emission spectrum as the second probe, but with a different quantum yield. Use of this latter configuration allows the determination of the concentration of the target sequence, as well as discrimination between the potential variants in the target sequence. Further, it allows one to determine the dissociation constant of the second and third probes to the target sequence. An exemplary scheme for analyzing genetic variants is as follows: a short stable probe is chosen from a universal library to be complementary to a non-variable region of the target nucleic acid sequence. This probe is labeled with a first fluorophore that has a first set of fluorescent characteristics, e.g., excitation and emission spectra, and fluorescent quantum yield. A second probe is chosen from a second universal library that is complementary to one of the genetic variants of the target sequence. The second probe is labeled with a second fluorophore that is different from the first fluorophore. A third probe is chosen from a third universal library that is complementary to the other genetic variant of the target sequence. The first fluorophore has a different emission spectrum from both the second and third fluorophores, while the second and third fluorophores differ from each other by virtue of the quantum yields. As a result, single molecules are distinguishable from each other with respect to fluorescent signals. The first probe is used to quantify the target and can be used to quantify the dissociation constants of the two allele specific probes under the set of experimental conditions used, and thereby identify the perfectly matching probe in the mixture.

III. Overall System Architecture

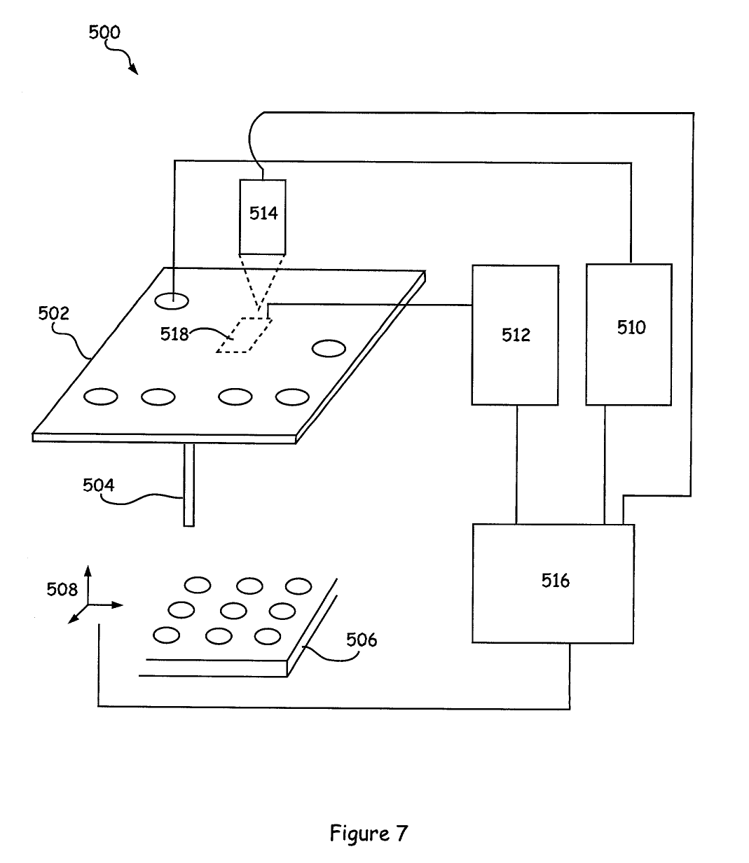

Systems are also provided for carrying out the above-described methods. One example of an overall system configuration is illustrated in FIG. 7. As shown, overall system 500 includes microfluidic channel network/device 502 (represented in a generic fashion, as shown), in which the reactions of interest are carried out. The microfluidic device includes sample accession capillary or “sipper” 504 for drawing different reagents into the channels of the device. The system also includes reagent library or substrate 506 from which different reagents may be accessed. In the context of the present invention, reagent library 506 typically includes one or both of the universal library of hybridization probes, as well as a collection of different patient specific reagents or other locus specific reagents, e.g., amplification primers, typically on separate substrates. In order to maximize the efficiency of the system, the different reagent libraries are provided immobilized or dried upon the substrate, e.g., as described in U.S. Pat. No. 6,042,709, in relatively high density, e.g., greater than 10,000 different reagents or reagent locations per substrate. The reagent substrates are typically disposed on an electronically controlled x-y-z translation stage (represented by arrows 508), which permits accession of the various reagents by capillary element 504 on microfluidic device 502. The microfluidic channel network is operably coupled to flow controller 510 which moves material into and through the various channels of the device in accordance with a prescribed flow profile. Thermal controller 512 is also operably coupled to thermal control region 518 on the device (whether a resistive heater incorporated in the device, or as a separate heating element placed adjacent to the device) in order to carry out the various thermal manipulations required for the overall analysis, e.g., thermal cycling for amplification and temperature sweeping for discrimination.

Detector 514 is provided in sensory communication with the appropriate portions or detection zones of the channel network, in order to ascertain the results of the particular discrimination reaction that is carried out. Controllers, 508, 510 and 512, as well as detector 514 are all typically coupled to computer or processor 516, that receives data from the detector and subjects that data to appropriate analysis, providing a reasonably user friendly output for observation for the results. The computer also instructs the operation of the various controllers in accordance with preprogrammed instructions, so as to access appropriate reagents and direct those reagents, as well as others housed on the device, into appropriate portions of the channel network to carry out the reactions of interest.

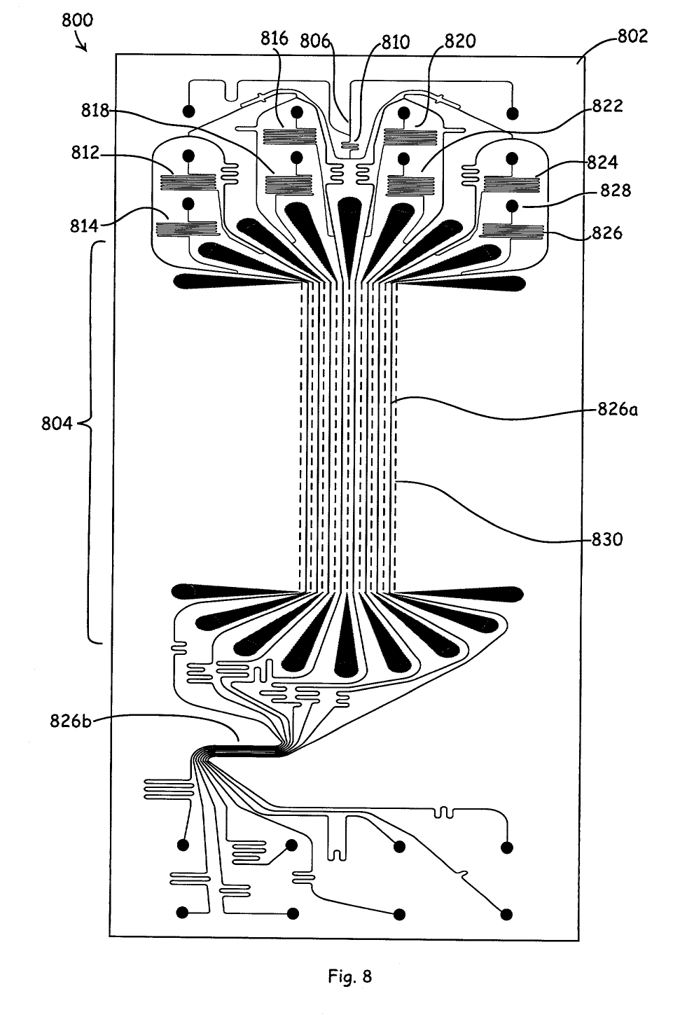

As noted above, in preferred aspects, the methods and systems of the invention employ microfluidic channel networks in carrying out at least a portion of the overall assay. One schematic example of a microfluidic channel network useful in accordance with the present invention is illustrated in FIG. 8. As shown, overall device 800 includes body structure 802, that includes channel network 804 disposed therein. Device 800 also includes external sample accession capillary element or pipettor 806, which is used to sip reagents or other materials into the channel network from sources external to the device itself, e.g., multiwell plates. As shown, channel network 804 includes common channel 810 that receives the materials drawn into the network from the pipettor element. This common channel is fluidly connected to a plurality of separate analysis channels 812-826. The analysis channels are used to perform different assays on separate aliquots of the same material drawn into the channel network. For example, where patient specific reagents, e.g., genomic DNA is drawn into the channel network from an external source, then in each channel, an aliquot of that material is subjected to the analytical operation to screen for a specific polymorphic genetic marker or SNP, by introducing different locus specific reagents, e.g., probes and primers, into different analysis channels. However, where the locus specific reagents are introduced into the channel network from an external source, then different patient specific reagents may be introduced and screened in different analysis channels. The number of different analysis channels typically depends upon the desired rate of throughput for the overall system, and for each channel network incorporated in that system. Typically, a given channel network will include between about 1 and about 20 separate analysis channels, and preferably, between 5 and 15, with 8 to 12 analysis channels being most preferred.

Each analysis channel typically is fluidly connected to a source of reagents, e.g., reservoir 828, that may include either locus or patient specific reagents. In addition, each analysis channel typically includes at least one, and often times, several heating zones, e.g., regions 826a and 826b, for carrying out different desired operations within the analysis channel. By way of example, within region 812a, an amplification reaction is optionally carried out to amplify the section of the patient's genomic DNA that includes the particular polymorphic locus. This is generally accomplished by combining the patient's DNA with appropriate amplification reagents, e.g., primers, polymerase and dNTPs, and thermally cycling the contents of the channel, e.g., within region 812a, through a melting, annealing and extension process, until sufficient amplified product has been produced.

As noted above, in certain preferred aspects, heating region 812a is heated using electrical current supplied by electrodes disposed in electrical contact with opposite ends of the heating region. Heat is then generated by applying current through that region until the desired temperature is achieved. This process is described in detail in U.S. Pat. No. 5,965,410, which is incorporated herein by reference in its entirety for all purposes. Similarly, preferred electrode and channel configurations for such Joule heating are described in U.S. patent application Ser. No. 60/269245, filed Feb. 15, 2001, which is incorporated herein by reference in its entirety for all purposes. Alternatively, conventional heating mechanisms may be employed, including the use of an external heating element, e.g., a hot plate or Peltier device, placed adjacent to the heating region to cycle the temperature therein, or a resistive heater deposited upon the device and near or within the heating region of the channel. Examples of resistive heaters include those described in U.S. Pat. No. 6,132,580, which is incorporated herein by reference in its entirety for all purposes.

FIG. 8 illustrates one embodiment of resistive heaters for temperature control of the multiple analysis channels. The resistive heaters comprises multiple thin resistive metal films, shown as dotted lines e.g. 830a, deposited on both sides of each analysis channel. The resistive heaters are connected to electrical leads for the application of a voltage across the metal film. Heat from the metal films heats the content of the channel disposed between two metal films. Temperature sensors are incorporated into the devices of the invention for measuring the temperature within the heated regions of the channel network. In the embodiment shown in FIG. 8, the temperature sensors comprise resistance thermometers which include material having an electrical resistance proportional to the temperature of the material. Other temperature sensors suitable for use with the devices of the present invention include thermistors, IC temperature sensors, quart thermometers and the like. See, Horowitz and Hill, The Art of Electronics, Cambridge University Press 1994 (2^{nd} Ed. 1994).

### EXAMPLES

### Example 1

Use of Two Anchor Two Probe Combinations from a Universal Library of Probes and Anchors.

Probe and Anchor Selection:

Anchor 1 and 2: 6 mers, 3′ labeled with a Dark Quencher.

Probe 1: 6 mer Oligo that was AT rich; 5′ labeled with Fam.

Probe 2: 6 mer Oligo; 5′ labeled with Vic (Z38).

Reagent Concentrations: 10 nM Tris; 50 nM KCl pH 8.4.

Probe and Anchor Conc: 1000 nM.

Target Conc: 200 nM of Synthetic Target.

NaCl Conc: 1M.

Instrument: ABI 7900.

Detection Temperature Range: 4° C. to 94° C.

Results: Specific signals were obtained for the Fam and Vic dyes. There was no background due to the Dark Quencher. The discrimination between the mismatch Target and the Target was easily detectable using FRET. The melting temperature of the hybrids(Tm) for each of the probes were reproducible between several runs.

FIG. 5 shows a plot illustrating the discrimination between the matched and mismatched hybridizations.

### Example 2

Mutation Detection by Fluorescence Polarization Using LNA Probes

Probe Selection:

Probe 1: 6 mer Oligo Rhodamine labeled Rho-GTCGCC.

Probe 2: 6 mer Oligo; Rhodamine labeled Rho-GTCACC.

Reagent Concentrations: 50 mM HEPES Buffer; 50 nM KCl pH 7.5.

Probe Conc: 50 nM.

Target Conc: 50 nM.

NaCl Conc: 100 mM.

Enzyme: T7 gene 6 exonuclease at 1 unit/μL

Instrument: Agilent 2100 BioAnalyzer.

Fluorescence Spectrophotometer (Fluoromax-2 or Fluorolog-3, JY Horiba, Edison, N.J.).

Detection Temperature Range: 12-80 degrees celsius

Target Sequence:

aagaggacttccacgtggaccaggT/Cgaccaccgtgaaggtgcctatgatgaagcgtt

Experiment Description: Human genomic DNA was amplified using PCR to yield a 107 base pair product containing the SNP. The forward primer contained five phosphorotioate residues at its 5′ end to ensure upper strand survival during exonuclease digestion. PCR reactions were performed with four different templates: 1) no-template control 2) human genomic DNA 231 (homozygous C) 3) human genomic DNA 207 (heterozygous C/T) and 4) human genomic DNA 208 (homozygous T). Amplification results were observed with the Agilent BioAnalyzer.

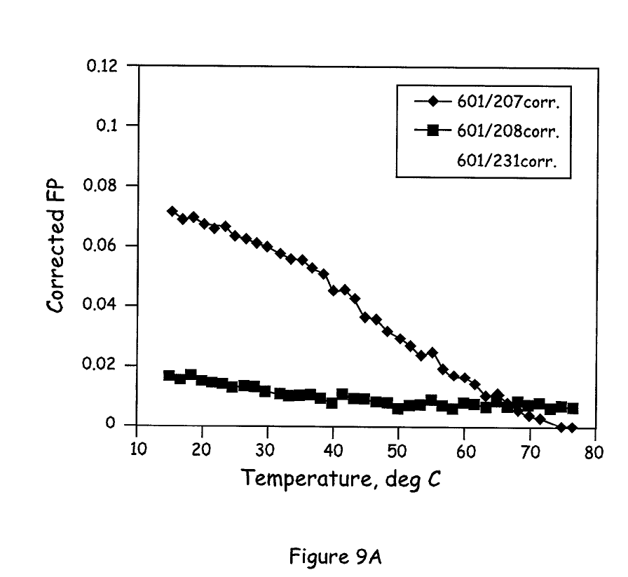

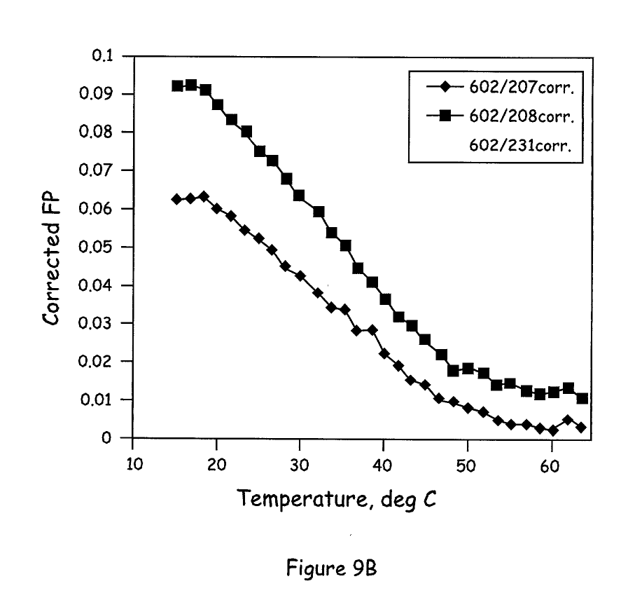

Real-Time detection: Post amplification detection was achieved by real-time detection as well as a melting curve. For real-time detection, PCR reaction prouducts were transferred to fluorometer cuvettes and 50 mM of the respective LNA probes were added to each. The fluorescence polarization as a function of time was recorded before and after the addition of the exonuclease. The exonuclease digestion generates single-stranded DNA target and the LNA probe comprising the interrogation base is subsequently allowed to hybridize to the target sequence. FIG. 9 illustrates the increase in fluorescence polarization where there is a perfect match between the LNA probe and the target sequence.

Melting Curve detection: PCR reaction products were digested for 10 minutes with T7 gene 6 exonuclease and then mixed with 50 nM probe in fluorometer cuvette. The cuvettes were stored in a water bath where the temperature was gradually increased to allow for the detection of the melting temperature for the duplexes.

Results: The real-time detection results correlated with the melting curve detection indicating that the two LNA probes exhibited excellent discrimination, displaying typical melting behavior only where there was a perfect match between the probe and the target.

Although described with reference to specific examples, a wide variety of variations can be made to the described components without departing from the scope of the invention described herein. For example, any of the devices, systems or libraries can be provided as kits, e.g., including the devices, systems or libraries and appropriate containers, packaging material, instructions in the use of the devices, systems or libraries, or the like. In addition, the invention provides for the use of any of the components herein, e.g., in the practice of any of the methods herein. All patents, patent application and publications cited herein are incorporated by reference in their entirety for all purposes, as if each were specifically indicated to be incorporated by reference for all purposes.

What is claimed is:

1. A method of detecting a target nucleic acid in a sample, the method comprising:
providing at least a first and second group of nucleic acid probes, the first group of probes comprising at least 10% of all possible nucleic acid probes having x number of nucleotides and the second group of probes including at least 10% of all possible nucleic acid sequences having at least y number of nucleotides;
hybridizing at least a first probe from the first group and at least a second probe from the second group to target nucleic acid; and,
detecting the hybridization by a non-Sanger detection step, thereby detecting the target nucleic acid.

2. The method of claim 1, wherein the first and second probes are substantially proximal when hybridized to the target nucleic acid, wherein proximity of the first probe to the second probe stabilizes binding of at least one of the first and second probes.

3. The method of claim 1, further comprising ligating the first and second probes.

4. The method of claim 1, wherein x or y is an integer between about 5 to about 10, inclusive.

5. The method of claim 1, wherein x or y is an integer between about 6 to about 9, inclusive.

6. The method of claim 1, wherein x or y is 7.

7. The method of claim 1, wherein x=y.

8. The method of claim 1, wherein x or y is an integer between about 5 and about 18, inclusive.

9. The method of claim 1, wherein x or y is an integer between about 7 and about 12, inclusive.

10. The method of claim 1, wherein x or y is 10.

11. The method of claim 1, wherein the first and second groups are components of a single physical group.

12. The method of claim 1, wherein the first and second groups are components of a plurality of physical groups.

13. The method of claim 1, wherein the first or second probes comprise at least one promiscuous base.

14. The method of claim 13, wherein the at least one promiscuous base is selected from a group consisting of: inosine, and azidothimidine.

15. The method of claim 1, wherein the first or second groups comprise one or more of: a nucleobase analog, a sugar analog, or an internucleotide analog.

16. The method of claim 1 or 15, wherein the first or second groups comprise at least 60% of all possible nucleic acid probe sequences having x or y nucleotides.

17. The method of claim 1 or 15, wherein the first or second groups comprise at least 70% of all possible nucleic acid probe sequences having x or y nucleotides.

18. The method of claim 1 or 15, wherein the first or second groups comprise at least 80% of all possible nucleic acid probe sequences having x or y nucleotides.

19. The method of claim 1 or 15, wherein the first or second groups comprise at least 90% of all possible nucleic acid probe sequences having x or y nucleotides.

20. The method of claim 1 or 15, wherein the first or second groups comprise at least 95% of all possible nucleic acid probe sequences having x or y nucleotides.

21. The method of claim 1, wherein the first or second group further comprise probes of length w, wherein w is not equal to x or y.

22. The method of claim 1, wherein the first or second group further comprise probes of length w, wherein w is not equal to x or y, wherein a Tₘ of the probes of the first or second group, or both the first and second group, are selected to be approximately equal.

23. The method of claim 15, wherein the nucleobase analogs include covalently bound minor groove binders or intercalators that enhance hybridization avidity or specificity of the first or second probes to the target nucleic acid.

24. The method of claim 15, wherein the internucleotide analogs comprise one or more of: a phosphate ester analog, or a non-phosphate oligonucleotide analog.

25. The method of claim 24, wherein the non-phosphate oligonucleotide analog is a PNA.

26. The method of claim 24, wherein the phosphate ester analogs are selected from a group consisting of conformationally restricted nucleotides, alkyl phosphonates, phosphoroamidates, alkylphosphotriesters, phosphorothioates, and phosphorodithioates.

27. The method of claim 1, wherein the first probe is labeled with a fluorescent reporter moiety, and the second probe is labeled with a quencher moiety, such that upon hybridization of the probes with the target nucleic acid, fluorescence of the reporter moiety is quenched, thereby reducing fluorescence of the reporter moiety.

28. The method of claim 27, wherein the reporter and quencher moieties are selected from pairs of reporters and quenchers comprising: terbium chelate and TRITC (tetrarhodamine isothiocyanate), europium cryptate and Allophycocyanin, DABCYL and EDANS, Fluorescein and Tetramethylrhodamine, IAEDANS and Fluorescein, Fluorescein and Fluorescein, BODIPY FL and BODIPY FL, and Fluorescein and QSY 7 dye.

29. The method of claim 1, wherein the first probe is labeled with a fluorescent reporter moiety at one of its termini, and the second probe is labeled with a quencher moiety at one of its termini, such that hybridization of the first and second probes with the target nucleic acid causes an increase in fluorescence emission.

30. The method of claim 29, or 29, wherein detecting hybridization of the first or second probes comprises fluorescence resonance energy transfer (FRET) detection.

31. The method of claim 29, or 29, wherein the fluorescent reporter moiety is selected from a group consisting of: Xanthene dyes, Cyanine dyes, and Metal-Ligand Complexes.

32. The method of claim 29, or 29, wherein the first and second probes comprise a FRET pair.

33. The method of claim 1, further comprising at least a third group of nucleic acid probes comprising at least 10% of all nucleic acid probe sequences having z nucleotides, wherein the method further comprises hybridizing at least a third probe from the third group to the target nucleic acid substantially proximal to at least one of the first and second probes.

34. The method of claim 33, further comprising ligating the first, second and third probes together with a ligase.

35. The method of claim 33, wherein the first and second probes are hybridized to the target nucleic acid in a mixture comprising a buffer.

36. The method of claim 33, wherein the probes from the second and third groups are each labeled with a label.

37. The method of claim 36, wherein the label of the probes from the second group is different from the label of the probes from the third group.

38. The method of claim 37, wherein the at least one probe from the second group is labeled with a fluorescent reporter dye at one of its termini, and the at least one probe from the third group is labeled with a quencher molecule at one of its termini, such that upon hybridization of the probes from the first, second and third groups with the sample, the fluorescence of the reporter dye is quenched so as to cause a reduction in fluorescence emission of the reporter dye.

39. The method of claim 1, wherein the detecting step comprises observing the fluorescence of the hybridized probes while varying temperature over a range of temperatures.

40. The method of claim 39, wherein the range of temperatures during which fluorescence is observed is from about 0° C. to about 60° C.

41. The method of claim 1, wherein the target nucleic acid comprises a polymorphic variant sequence.

42. The method of claim 41, wherein the first probe is fully complementary to the polymorphic variant sequence and the second probe hybridizes substantially adjacent to the polymorphic variant sequence.

43. The method of claim 41, wherein detecting the target nucleic acid comprises detecting the polymorphic variant sequence.

44. The method of claim 41, wherein the target nucleic acid is derived from a patient, an animal, a plant, a bacteria, a fungi, an archae, a cell, a tissue, or an organism.

45. The method of claim 41, wherein the target nucleic acid is derived from a bacteria, archae, plant, non-human animal, cell, fungi, or non-human organism and the method further comprises selecting the a bacteria, archae, plant, non-human animal, cell, fungi, or non-human organism based upon detection of the target nucleic acid.

46. The method of claim 41, wherein the target nucleic acid is derived from a patient.

47. The method of claim 46, wherein the method further comprises selecting a treatment, diagnosing a disease, or diagnosing a genetic predisposition to a disease, based upon detection of the target nucleic acid.

48. The method of claim 1, wherein the first and second probes are hybridized to the target nucleic acid in a mixture comprising a buffer.

49. A method for detecting a polymorphic variant in a polymorphic nucleic acid sequence, the method comprising:
flowing a mixture comprising a polymorphic nucleic acid sequence, at least two probes and a buffer into an analysis channel, one of the at least two probes being complementary to a portion of the polymorphic nucleic acid sequence comprising the polymorphic variant, and the other probe being complementary to a substantially adjacent portion of the polymorphic nucleic acid sequence; and,
detecting hybridization of at least one of the at least two probes to determine the identity of the polymorphic variant in the polymorphic nucleic acid sequence by varying temperature within a detection region located at a position along a length of the analysis channel.

50. The method of claim 48 or 49, wherein the mixture comprises a salt in a concentration from a range of about 0.2M to about 2M.

51. The method of claim 48 or 49, wherein the salt concentration is in the range of about 0.5M to about 1.5M.

52. The method of claim 48 or 49, wherein the salt concentration is in the range of about 0.8M to about 1.2 M.

53. The method of claim 48 or 49, wherein the salt concentration is about 1M.

54. The method of claim 49, wherein the detecting step comprises measuring a signal intensity resulting from hybridization of the hybridizing probes and the target nucleic acid.

55. The method of claim 49, wherein the method comprises providing the analysis channel in a microfluidic device, wherein the analysis channel comprises one or more detection regions and one or more temperature control regions.

56. A method of detecting a target nucleic acid, the method comprising:
flowing a mixture comprising the target nucleic acid in an analysis channel;
flowing at least a first probe and a second probe into the analysis channel;
hybridizing a first probe to the target nucleic acid;
hybridizing a second probe to the target nucleic acid, wherein the second probe hybridizes to the target nucleic acid substantially adjacent to the first probe, and wherein hybridization of the second probe stabilizes hybridization of the first probe; and,
detecting hybridization of the first probe by a non-Sanger detection step.

57. The method of claim 56, wherein the target nucleic acid is derived from a patient, an animal, a plant, a bacteria, a fungi, an archae, a cell, a tissue, or an organism.

58. The method of claim 56, wherein the target nucleic acid comprises a polymorphic sequence.

59. The method of claim 56, wherein the first or second probe comprises a fluorescent label.

60. The method of claim 59, wherein the first probe comprises a first label and the second probe comprises a second label, wherein the first label is quenched by proximity to the second label.

61. The method of claim 60, wherein the first probe and the second probe collectively comprise a FRET label pair.

62. The method of claim 56, wherein the first or second probe is provided from at least one probe set comprising at least 10% of all possible nucleic acids of a selected type for a selected length, wherein the selected length is at least about 5 probe monomers.

63. The method of claim 56, wherein the first probe and the second probe are independently selected from at least two groups of probes, each comprising at least 10% of all possible nucleic acids of a selected type for a selected length, wherein the selected length for each of the two groups is at least about 5 probe monomers.

64. The method of claim 56, wherein the first probe and the second probe are independently selected from at least two groups of probes, each comprising at least 10% of all possible nucleic acids of a selected type for a selected length, wherein the selected length for each of the two groups is at least about 5 probe monomers, the method further comprising hybridizing a third probe to the target nucleic acid, wherein the third probe is selected from a third group of probes comprising at least 10% of all possible nucleic acids of a selected type for a selected length, wherein the selected length is at least about 5 probe monomers.

65. The method of claim 64, wherein the first, second or third group of probes comprises at least two probe sizes.

66. The method of claim 65, wherein the at least two probe sizes are selected to have an approximately equal Tₘ.

67. The method of claim 62, wherein the probe monomers comprise one or more of: a nucleotide, or a PNA monomer.

68. The method of claim 56, wherein the first probe is provided from at least one probe set comprising at least 10% of all possible nucleic acids of a selected type for a selected length, wherein the selected length is at least 5 probe monomers and the second probe is provided from at least a second probe set comprising at least 10% of all possible nucleic acids of a selected type for a selected length, wherein the selected length is at least about 5 probe monomers.

69. The method of claim 56, wherein detection of hybridization of the first probe comprises detecting FRET between a first label on the first probe and a second label on the second probe.

70. The method of claim 56, wherein the first and second probes are components of a single physical group.

71. The method of claim 56, wherein the first and second probes are components of multiple physical groups.

72. The method of claim 56, wherein the mixture comprises a salt in a concentration from a range of about 0.2M to about 2M.

73. The method of claim 56, wherein the salt concentration is in the range of about 0.5M to about 1.5M.

74. The method of claim 56, wherein the salt concentration is in the range of about 0.8M to about 1.2 M.

75. The method of claim 56, wherein the salt concentration is about 1M.

76. A set of nucleic acid probes for detection of a target nucleic acid sequence in a sample, comprising:
at least two groups of nucleic acid probes, a first of the at least two groups comprising at least 10% of all possible nucleic acid probe sequences having x nucleotides, and a second of the at least two groups comprising at least 10% of all possible nucleic acids having y nucleotides;
wherein a plurality of members of each of the first and second groups are labeled.

77. The set of claim 76, wherein labels of the members of the first group interact with labels of the second group, when the labels are in proximity to one another.

78. The set of claim 76, wherein hybridization of a member of the first group to the target nucleic acid stabilizes hybridization of a member of the second group.

79. The set of claim 76, wherein the two groups include at least 60% of all possible nucleic acid probe sequences for nucleic acid probes of length x or y.

80. The set of claim 76, wherein the two groups include at least 70% of all possible nucleic acid probe sequences for nucleic acid probes of length x or y.

81. The set of claim 76, wherein the two groups include at least 80% of all possible nucleic acid probe sequences for nucleic acid probes of length x or y.

82. The set of claim 76, wherein the two groups include at least 90% of all possible nucleic acid probe sequences for nucleic acid probes of length x or y.

83. The set of claim 76, wherein the two groups include at least 95% of all possible nucleic acid probe sequences for nucleic acid probes of length x or y.

84. The set of claim 76, wherein the probes of the first group comprise a first label and the probes of the second group comprise a second label.

85. The set of claim 84, wherein the first label comprises an acceptor FRET moiety and the second label comprises a donor FRET moiety.

86. The set of claim 85, wherein the acceptor moiety comprises a quencher moiety.

87. The set of claim 86, wherein the quencher is selected from a group consisting of: fluorophores, Dabsyl, Black-hole™, QSY™, and an Eclipse Dark Quencher.

88. The set of claim 85, wherein the donor is selected from a group consisting of: Xanthene dyes, Cyanine dyes, Metal-Ligand Complexes, Coumarin dyes, BODIPY dyes, and Pyrene dyes.

89. The set of claim 76, wherein at least one of the at least two groups further comprise a subset of probes having w number of nucleotides, wherein when present in the first set, w is not equal to x and when present in the second set w is not equal to y.

90. The set of nucleic acid probes of claim 76, wherein the first or second groups comprise probes of a length other than x or y, respectively.

91. The set of nucleic acid probes of claim 89, wherein w is an integer between about 1 and about 10, inclusive.

92. The set of nucleic acid probes of claim 89, wherein w is an integer between about 1 and about 8, inclusive.

93. The set of claim 76, further comprising at least a third group of nucleic acid probes comprising at least 10% of all nucleic acid probe sequences having z nucleotides.

94. The set of nucleic acid probes of claim 93, wherein z is an integer between about 5 and about 10, inclusive.

95. The set of nucleic acid probes of claim 76, wherein x is an integer between about 5 and about 10, inclusive.

96. The set of nucleic acid probes of claim 76, wherein x is an integer between about 6 and about 9, inclusive.

97. The set of nucleic acid probes of claim 76, wherein x is 7.

98. The set of nucleic acid probes of claim 76, wherein x=y.

99. The set of nucleic acid probes of claim 76, wherein y is an integer between about 5 and about 18, inclusive.

100. The set of nucleic acid probes of claim 76, wherein y is an integer between about 7 and about 12, inclusive.

101. The set of nucleic acid probes of claim 76, wherein y is 10.

102. The set of nucleic acid probes of claim 76, wherein the first and second groups are components of a single physical group.

103. The set of nucleic acid probes of claim 76, wherein the first and second groups are components of a plurality of physical groups.

104. A library of nucleic acids, comprising:
at least about 10% of all possible nucleic acids for a monomer length x, wherein x is greater than or equal to 5, wherein the nucleic acids comprise non-natural nucleic acid monomers.

105. The library of claim 104, wherein the nucleic acids of the library display greater avidity or specificity for a target nucleic acid than a corresponding natural nucleic acid.

106. The library of claim 104, wherein the nucleic acids of the library comprise one or more of: a PNA, an LNA, and a base-modified nucleic acid.

107. The library of claim 104, wherein the nucleic acids of the library comprise one or more of: a nucleobase analog, a sugar analog, or an internucleotide analog.

108. The library of claim 104, wherein the nucleic acids of the library comprise one or more labels.

109. The library of claim 108, wherein the labels of the library comprise one or more fluorescent, luminescent, or colorimetric labels.

110. The library of claim 108, wherein the labels of the library comprise one or more FRET pairs.

111. The library of claim 104, wherein at least 90% of the 10% comprise of one or more of: a PNA, an LNA, and a base-modified nucleic acid.

112. The library of claim 104, wherein at least 90% of the 10% consist of one or more of: a PNA, an LNA, and a base-modified nucleic acid.

113. The library of claim 104, comprising at least about 10% of all possible nucleic acids for a monomer length y, wherein y is greater than or equal to 5 and does not equal x, and wherein the nucleic acids comprise non-natural nucleic acid monomers.

114. The library of claim 113, comprising at least about 10% of all possible nucleic acids for a monomer length z, wherein z is greater than or equal to 5 and does not equal x or y, and wherein the nucleic acids comprise non-natural nucleic acid monomers.

115. The library of claim 104, wherein the members of the library are arranged in substantially separate pools.

116. The library of claim 104, wherein the members of the library are arranged in substantially overlapping pools.

117. The library of claim 104, wherein the members of the library are arranged dried on a solid surface in a re-hydrateable form.

118. The library of claim 104, wherein the members of the library are arranged in microtiter wells.

119. The library of claim 104, wherein the members of the library are arranged in a microfluidic system.

120. The library of claim 104, wherein the members of the library are arranged in a format accessible by a microfluidic system.

121. A genetic analysis system, comprising:
a vessel comprising a mixture, the mixture comprising a target nucleic acid;
a plurality of sources of nucleic acid probes, the plurality of sources each including probes of at least 10% of all possible nucleic acid probe sequences of length x or y; and,
a system for selectively delivering different probes from the plurality of sources of probes to the vessel, comprising:
system instructions which identify and select probes to be delivered to the vessel;
and a sampling system for sampling and delivering probes from the plurality of sources of probes to the vessel, wherein the vessel is a microfluidic device, and the system instructions select probes that are complementary to a region of interest on the target nucleic acid.

122. The system of claim 121, wherein the sampling system comprises a pipettor affixed to the microfluidic device.

123. The system of claim 121, wherein the sampling and delivering probes comprises delivering at least three nucleic acid probes from the plurality of sources of probes to the vessel.

124. The system of claim 121, wherein the nucleic acid probes comprise hybridizing probes and flanking sequences, the hybridizing probes comprising at least one interrogation base.

125. The system of claim 124, wherein at least one of the hybridizing probes comprises one or more of: nucleobase analogs, sugar analogs, or internucleotide analogs in its sequence.

126. The system of claim 125, wherein the nucleobase analogs include covalently bound minor groove binders, intercalators or other modifications for enhancing hybridization avidity or specificity of the nucleic acid probes.

127. The system of claim 125, wherein the nucleobase analogs include non-covalently bound minor groove binders selected from a group consisting of DAPI, and Hoeschst 33258.

128. The system of claim 125, wherein the internucelotide analogs comprise one or more of: a phosphate ester analog, or a non-phosphate oligonucleotide analog.

129. The system of claim 125, wherein the phosphate ester analogs are selected from a group consisting of alkyl phosphonates, phosphoroamidates, alkylphosphotriesters, phosphorothioates, and phosphorodithioates.

130. The system of claim 121, wherein the plurality of sources of nucleic acid probes comprise sources of at least 75% of all possible nucleic acid probe sequences of length x or y

131. The system of claim 121, wherein the plurality of sources of nucleic acid probes comprise sources of at least 85% of all possible nucleic acid probe sequences of length x or y.

132. The system of claim 121, wherein the plurality of sources of nucleic acid probes comprise sources of at least 95% of all possible nucleic acid probe sequences of length x or y.

133. The system of claim 121, wherein the vessel is in contact with a thermal element, whereby at least a region of the vessel is subjected to an increase or decrease in temperature.

134. The system of claim 121, wherein x or y is an integer between about 5 and about 10, inclusive.

135. The system of claim 121, wherein x or y is an integer between about 6 and about 9, inclusive.

136. The system of claim 121, wherein at least one of x or y is 7.

137. The system of claim 121, wherein at least one of x or y is 6.

138. The system of claim 121, further comprising a detector.

139. The system of claim 121, wherein the microfluidic device comprises at least two intersecting microscale channels wherein at least one of the at least two intersecting channels is an analysis channel.

140. The system of claim 139, wherein the analysis channel is subjected to an increase or decrease in temperature.

141. The system of claim 121, wherein the mixture comprises salt in a concentration from about 0.2M to about 2M.

142. The system of claim 141, wherein the salt concentration is in a range of from about 0.5M to 1.5M.

143. The system of claim 141, wherein the salt concentration is in a range of from about 0.8M to 1.2 M.

144. The system of claim 141, wherein the salt concentration is 1M.
